(19) United States
(12) Patent Application Publication
(10) Pub. No.: US 2025/0186446 A1
(43) Pub. Date: Jun. 12, 2025

(54) ORGANIC COMPOUNDS

(71) Applicant: INTRA-CELLULAR THERAPIES, INC., New York, NY (US)

(72) Inventors: Maud VOISIN, New York, NY (US); Stephen GODING, Suffern, NY (US); Gretchen L. SNYDER, New York, NY (US); Robert E. DAVIS, San Diego, CA (US)

(21) Appl. No.: 18/846,037

(22) PCT Filed: Mar. 11, 2023
(86) PCT No.: PCT/US2023/064202

§ 371 Date: Sep. 11, 2024

Related U.S. Application Data
(60) Provisional application No. 63/479,938, filed on Jan. 13, 2023.
(60) Provisional application No. 63/269,209, filed on Mar. 11, 2022.

(51) Int. Cl. A61K 31/519; A61K 39/00; A61K 39/395; A61P 35/00

(52) U.S. Cl. CPC A61K 31/519; A61K 39/3955; A61P 35/00; A61K 2039/505

(57) ABSTRACT

Please add the following heading and paragraph on a separate sheet, after the claims: The disclosure relates to the use of phosphodiesterase 1 (PDE1) inhibitors alone or in combination with immune checkpoint inhibitor therapies for the treatment of breast cancer, including for promoting antitumor immunity and mitigating the side effects (i.e., inflammatory-related adverse events) associated with checkpoint inhibitor therapies.

### RELATED APPLICATIONS

This application claims the benefit of U.S. Provisional Application No. 63/269,209, filed Mar. 11, 2022; and U.S. Provisional Application No. 63/479,938, filed Jan. 13, 2023. The entire contents of each of these applications is incorporated herein by reference.

### FIELD OF DISCLOSURE

The field relates to the use of phosphodiesterase 1 (PDE1) inhibitors alone or in combination with immune checkpoint inhibitor therapies for the treatment of breast cancer, including for promoting antitumor immunity and mitigating the side effects (i.e., inflammatory-related adverse events) associated with checkpoint inhibitor therapies.

### BACKGROUND OF THE DISCLOSURE

Breast cancer is cancer that develops from breast tissue. About 10 to 15% of breast cancers are classified as triple-negative breast cancer (TNBC). TNBC is a complex and aggressive subtype of breast cancer lacking estrogen receptor, progesterone receptor, and HER2. TNBC is the one of the most challenging breast cancers to treat because TNBC does not respond to drugs that target estrogen receptor, progesterone receptor, or HER2. Consequently, there's a substantial need for better treatment options for TNBC.

Immunotherapy, including checkpoint inhibitors, has revolutionized cancer treatment and are effective for many breast cancer patients. Immunotherapy helps the patient's immune system to prevent the growth of cancers. As part of its normal function, the immune system detects and destroys abnormal cells and can also prevent or curb the growth of many cancers. However, cancer cells have ways to evade immune responses. Immune activation is primarily T-cell mediated and regulated by stimulatory, co-stimulatory, and inhibitory (checkpoint) signals. When T-cells encounter a self-cell, there are important receptor-ligand interactions that provide a check on activation, so that the immune cells do not attack the body's normal cells. Cancer cells have genetic and epigenetic alterations which can result in antigen expression that can elicit an immune activation, but cancer cells can also exploit immune checkpoint interactions such as PD-1/PD-L1 and CTLA4/B7-1/B7-2 to deactivate the immune cells, rendering the immune system ineffective to destroy the cancer. Immune checkpoint inhibitors have been effective in many patients suffering from various types of cancers, as they allow destruction of the cancers by the patient's own immune system. Unfortunately, some patients do not benefit from these therapies and development of resistance might lead to cancer progression in patients with primary clinical responses. Therefore, resistance to checkpoint inhibitor (e.g., PD-1/PD-L) blockade remains a significant challenge and impedes their broader application.

Furthermore, immunotherapy-related adverse events can limit the use of checkpoint blockade therapy and can result in serious adverse outcomes. Blocking the immune checkpoints can allow the immune system to attack normal tissue. This leads to inflammatory conditions such as dermatitis, colitis, arthritis, nephritis, myositis, polymyalgia-like syndromes, and cytokine release syndrome (CRS) caused by a large, rapid release of cytokines into the blood from immune cells affected by the immunotherapy. These side effects can be very serious and occasionally fatal. Thus, in spite of its considerable benefit in patients with cancer, immune checkpoint blockade can be limited by the occurrence of immunotherapy-related adverse events.

Eleven families of phosphodiesterases (PDEs) have been identified but only PDEs in Family L, the Ca2+/calmodulin-dependent phosphodiesterases (CaM-PDEs), which are activated by Ca2+/calmodulin, have been shown to mediate the calcium dependent cyclic nucleotide (e.g., cGMP and cAMP) signaling pathways. The three known CaM-PDE genes, PDE1A, PDE1B, and PDE1C, are all expressed in central nervous system tissue. PDE1A is expressed in the brain, lung and heart. PDE1B is primarily expressed in the central nervous system, but it is also detected in monocytes and neutrophils and has been shown to be involved in inflammatory responses of these cells. PDE1C is expressed in olfactory epithelium, cerebellar granule cells, striatum, heart, vascular smooth muscle and tumor cells. PDE1C has been demonstrated to be a major regulator of smooth muscle proliferation in human smooth muscle. Cyclic nucleotide phosphodiesterases down-regulate intracellular cAMP and cGMP signaling by hydrolyzing these cyclic nucleotides to their respective 5′-monophosphates (5′AMP and 5′GMP), which are inactive in terms of intra-cellular signaling pathways. Both cAMP and cGMP are central intracellular second-messengers and they play roles in regulating numerous cellular functions. PDE1A and PDE1B preferentially hydrolyze cGMP over cAMP, while PDE1C shows approximately equal cGMP and cAMP hydrolysis.

There is a substantial need for new therapies which complements and enhances checkpoint inhibitor therapies, and a need for a safe and selective strategy for mitigating the serious side effects (i.e., inflammatory-related adverse events) associated with checkpoint inhibitor therapies.

### SUMMARY OF THE DISCLOSURE

In an aspect, the disclosure provides a method of treating breast cancer comprising administering a pharmaceutically acceptable amount of a PDE1 inhibitor alone or in combination with a pharmaceutically acceptable amount of an immune checkpoint inhibitor to a subject in need thereof. In some embodiments, the breast cancer is triple-negative breast cancer (TNBR), which is estrogen receptor-negative, progesterone receptor-negative, and HER2-negative. In certain embodiments, the TNBC is a high-risk early stage TNBC. In another embodiments, the treatment is an adjuvant treatment after TNBC is removed by surgery. In yet another embodiment, the subject has locally recurrent unresectable or metastatic TNBC whose tumors express PD-L1, e.g., Combined Positive Score (CPS)≥1 as determined by an FDA approved test, wherein CPS is the number of PD-L1 staining cells (tumor cells, lymphocytes, macrophages) divided by the total number of viable tumor cells, multiplied by 100.

In some embodiments, the immune checkpoint inhibitor is selected from one or more of inhibitors of CTLA-4, PD-1 and/or PD-L1. In certain embodiments, the immune checkpoint inhibitor is an is an inhibitor of PD-1, e.g., anti-PD-1 antibody. In some embodiments, the PDE1 inhibitor is a PDE1 inhibitor of Formulas I, Ia, II, III, IV, V, and/or VI described hereinbelow in free or pharmaceutically acceptable salt form. In some embodiments, the PDE1 inhibitor is Compound A in free or pharmaceutically acceptable salt form or Compound B in free or pharmaceutically acceptable salt form.

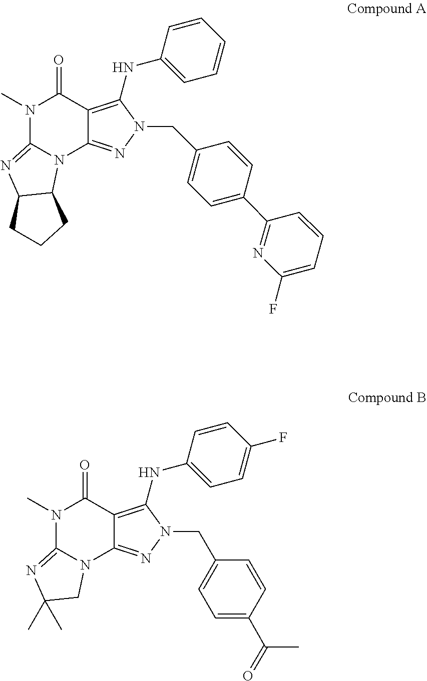 [SMILES: CN1C(=O)c2c(nn(Cc3ccc(-c4cccc(F)n4)cc3)c2Nc2ccccc2)N2C1=N[C@@H]1CCC[C@@H]12 ; CC(=O)c1ccc(Cn2nc3c(c2Nc2ccc(F)cc2)C(=O)N(C)C2=NC(C)(C)CN23)cc1]

In another aspect, the disclosure provides a method of prophylaxis or mitigation of a disease, disorder or adverse effect consequent to administration of an immune checkpoint inhibitor therapy to a subject suffering from breast cancer, the method comprising reducing the amount of checkpoint inhibitor administered to the subject and administering a pharmaceutically acceptable amount of a PDE1 inhibitor in combination with the immune checkpoint inhibitor therapy to the subject.

In another aspect, the disclosure provides a pharmaceutical combination therapy comprising a pharmaceutically acceptable amount of a PDE1 inhibitor and a pharmaceutically acceptable amount of an immune checkpoint inhibitor for use in the method of treating breast cancer or for prophylaxis or mitigation of a disease, disorder or adverse effect consequent to administration of a checkpoint inhibitor therapy.

### BRIEF DESCRIPTION OF THE DRAWINGS

FIG. 1 shows mean volume of E0771 tumors from mice treated with Compound A in diet at 900 ppm, anti-PD1 10 mg/kg, or combination treatment with Compound A (900 ppm)+anti-PD1 (10 mg/kg). n=6-7/group. *P<0.05, ns indicates no statistical difference.

FIG. 2 shows individual growth curve (volume) of E0771 tumors from mice treated with Compound A in diet at 900 ppm, anti-PD1 10 mg/kg, or combination treatment with Compound A (900 ppm)+anti-PD1 (10 mg/kg).

FIG. 3 shows mean tumor weight of E0771 tumors on harvest day from mice treated with Compound A in diet at 900 ppm, anti-PD1 10 mg/kg, or combination treatment with Compound A (900 ppm)+anti-PD1 (10 mg/kg). n=6-7/group. *P<0.05.

FIGS. 4A and B show flow cytometry analysis of E0771 tumors in mice treated with Compound A in diet at 900 ppm, anti-PD1 10 mg/kg, or combination treatment with Compound A (900 ppm)+anti-PD1 (10 mg/kg). FIG. 4A shows relative proportions of CD45 cells and macrophages in E0771 tumors. FIG. 4B shows the M1/M2 ratio of macrophages in E0771 tumors. n=6-7/group. t-test; *P<0.05.

FIG. 5 show flow cytometry analysis of E0771 tumors in mice treated with Compound A in diet at 900 ppm, anti-PD1 10 mg/kg, or combination treatment with Compound A (900 ppm)+anti-PD1 (10 mg/kg). FIG. 5 shows relative proportions of T cells, CD8+ cells, CD4+ cells and NK cells in E0771 tumors. n=6-7/group. t-test; *P<0.05.

FIG. 6 shows a Volcano plot of gene expression comparison of Compound A (900 ppm)+anti-PD1 (10 mg/kg) tumors and (control) group. The volcano plot shows that 48 genes are downregulated and 136 genes are upregulated in Compound A (900 ppm)+anti-PD1 (10 mg/kg) tumors (fold-change<−1.5 or >1.5; P<0.05).

FIG. 7 shows pathways enriched in the genes differentially expressed in Compound A (900 ppm)+anti-PD1 (10 mg/kg) tumors (top) and transcription regulators associated with upregulated or downregulated genes in Compound A (900 ppm)+anti-PD1 (10 mg/kg) tumors (bottom).

FIG. 8 shows mean volume of 4T1 tumors from mice treated with Compound A in diet at 300 ppm or 900 ppm, anti-PD1 10 mg/kg, or combination treatment with Compound A (300 ppm or 900 ppm)+anti-PD1 (10 mg/kg). n=7-8/group. *p<0.05; **p<0.01; and ***p<0.001, ns indicates no statistical difference.

FIG. 9 shows individual growth curve (volume) of 4T1 tumors from mice treated with Compound A in diet at 300 ppm or 900 ppm, anti-PD1 10 mg/kg, or combination treatment with Compound A (300 ppm or 900 ppm)+anti-PD1 (10 mg/kg).

FIG. 10 shows mean tumor weight of 4T1 tumors on harvest day from mice treated with Compound A in diet at 300 ppm or 900 ppm, anti-PD1 10 mg/kg, or combination treatment with Compound A (300 ppm or 900 ppm)+anti-PD1 (10 mg/kg). n=7-8/group. *p<0.05; **p<0.01; and ***p<0.001, ns indicates no statistical difference.

FIG. 11 shows survival curves of mice treated with Compound A in diet at 300 ppm or 900 ppm, anti-PD1 10 mg/kg, or combination treatment with Compound A (300 ppm or 900 ppm)+anti-PD1 (10 mg/kg). n=7-8/group.

FIG. 12 shows mean volume of E0771 tumors from mice treated with Compound B in diet at 100 ppm, 300 ppm, or 900 ppm, anti-PD1 1 mg/kg, or combination treatment with Compound B (100 ppm, 300 ppm, or 900 ppm)+anti-PD1 (1 mg/kg). n=5-6/group. *P<0.05, ns indicates no statistical difference.

FIG. 13 shows individual growth curve (volume) of E0771 tumors from mice treated with Compound B in diet at 100 ppm, 300 ppm, or 900 ppm, anti-PD1 1 mg/kg, or combination treatment with Compound B (100 ppm, 300 ppm, or 900 ppm)+anti-PD1 (1 mg/kg).

FIG. 14 shows mean tumor weight of E0771 tumors on harvest day from mice treated with Compound B in diet at 100 ppm, 300 ppm, or 900 ppm, anti-PD1 1 mg/kg, or combination treatment with Compound B (100 ppm, 300 ppm, or 900 ppm)+anti-PD1 (1 mg/kg). n=5-6/group. *P<0.05; **P<0.01.

FIG. 15 shows mean volume of 4T1 tumors from mice treated with Compound B in diet at 100 ppm, 300 ppm or 900 ppm, anti-PD1 10 mg/kg, or combination treatment with Compound B (100 ppm, 300 ppm or 900 ppm)+anti-PD1 (10 mg/kg). n=7-9/group. *p<0.05; **p<0.01; and ***p<0.001, ns indicates no statistical difference.

FIG. 16 shows individual growth curve (volume) of 4T1 tumors from mice treated with Compound B in diet at 100 ppm, 300 ppm or 900 ppm, anti-PD1 10 mg/kg, or combination treatment with Compound B (100 ppm, 300 ppm or 900 ppm)+anti-PD1 (10 mg/kg).

FIG. 17 shows mean tumor weight of 4T1 tumors on harvest day from mice treated with Compound B in diet at 100 ppm, 300 ppm or 900 ppm, anti-PD1 10 mg/kg, or combination treatment with Compound B (100 ppm, 300 ppm or 900 ppm)+anti-PD1 (10 mg/kg). n=7-9/group. *p<0.05; **p<0.01; and ***p<0.001, ns indicates no statistical difference.

FIGS. 18A and B show flow cytometry analysis of 4T1 tumors in mice treated with Compound B in diet at 900 ppm, anti-PD1 10 mg/kg, or combination treatment with Compound B (900 ppm)+anti-PD1 (10 mg/kg). FIG. 18A shows relative proportions of CD45 cells and macrophages in 4T1 tumors. FIG. 18B shows the M1/M2 ratio of macrophages in 4T1 tumors. n=6-7/group. t-test; *P<0.05.

FIG. 19 show flow cytometry analysis of 4T1 tumors in mice treated with Compound B in diet at 900 ppm, anti-PD1 10 mg/kg, or combination treatment with Compound B (900 ppm)+anti-PD1 (10 mg/kg). FIG. 19 shows relative proportions of various T cells in 4T1 tumors. n=6-7/group. t-test; *P<0.05.

FIG. 20 shows a Volcano plot of gene expression comparison of Compound B (900 ppm)+anti-PD1 (10 mg/kg) tumors and (control) group. The volcano plot shows that 281 genes are downregulated and 708 genes are upregulated in Compound B (900 ppm)+anti-PD1 (10 mg/kg) tumors (fold-change<−1.5 or >1.5; P<0.05).

FIG. 21 shows pathways enriched in the genes differentially expressed in Compound B (900 ppm)+anti-PD1 (10 mg/kg) tumors (FIG. 21A) and transcription regulators associated with upregulated or downregulated genes in Compound B (900 ppm)+anti-PD1 (10 mg/kg) tumors (FIG. 21B).

### DETAILED DESCRIPTION OF THE DISCLOSURE

The inventors have previously shown that inhibition of PDE1 activity using the presently disclosed compounds can safely restore cAMP function in a wide spectrum of pathological conditions, including models of neurodegeneration and neuroinflammation, heart failure, pulmonary hypertension and peripheral inflammation and in humans with certain diseases. More recently, the inventors have shown that PDE1 inhibitors modulate immune cell function (microglia and macrophages) by altering cell migration and levels of key cytokines (mainly CCL2 and TNF-α). Recent evidence indicates that PDE1, particularly the PDE1C isoform, is over expressed in experimental tumor models such as melanoma, neuroblastoma, renal cell and colon carcinomas, and osteosarcoma. In addition, focal genomic over representation of PDE1C in Glioblastoma Multiforme (GBM) cells has been demonstrated. Genomic gain of PDE1C is associated with increased expression in GBM-derived cell cultures and is essential for driving cell proliferation, migration and invasion in cancer cells.

Many types of cancer cells over-express PDE1 activity, which is identified through various biomarkers, such as increased RNA expression, DNA copy number, PDE1 binding (PET or radio-isotope retention of PDE1 inhibitor molecules) or enzymatic activity. These cancer cells also exhibit low levels of cAMP, which can be increased by PDE1 inhibitors.

In the present invention, it has been found that PDE-1 inhibitors when administered alone or in combination with immune checkpoint inhibitor therapies promote antitumor immunity, leading to the growth inhibition of breast cancer. It has been found that PDE-1 inhibitors alone or in combination with a sub-effective amount of anti PD-1 antibody can inhibit the growth of breast cancer in mouse model of triple-negative breast cancer (TNBC). In contrast, tumor growth in mice treated with anti-PD-1 antibody alone is similar to isotype control. It has been further found that the combination treatment shifts the polarization of macrophages toward a more inflammatory phenotype in the tumor microenvironment. Without being bound by any theory, it is believed that PDE-1 inhibitors affect macrophage infiltration and polarization, thereby promoting antitumor immunity. The synergistic ability of PDE-1 inhibitors and immune checkpoint inhibitors to alter the tumor microenvironment and inhibit tumor growth may provide a means to expand the utility of immune checkpoint inhibitors to treatment-refractory tumors such as TNBC. Furthermore, this synergistic ability of PDE-1 inhibitors in combination with sub-effective immune checkpoint inhibitors may provide a mean to mitigate adverse effects consequent to administration of a checkpoint inhibitor therapy to a subject suffering from breast cancer by reducing the dose of checkpoint inhibitor administered to the patient.

### Compounds for Use in the Methods of the Disclosure

In one embodiment, the PDE1 inhibitors for use in the methods of treatment and prophylaxis described herein are selective PDE1 inhibitors.

### PDE1 Inhibitors

In one embodiment, the PDE1 inhibitors for use in the methods of treatment and prophylaxis described herein are compounds of Formula I:

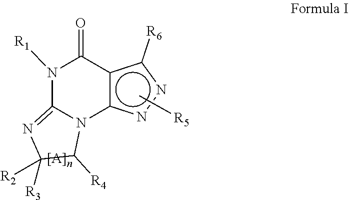

wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
   R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl; or R₅ is attached to one of the nitrogens on the pyrazolo portion of Formula I and is a moiety of Formula A

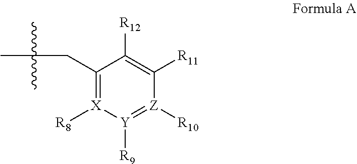

wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and (iv) R₆ is H, alkyl, aryl, heteroaryl, arylalkyl (e.g., benzyl), arylamino (e.g., phenylamino), heterarylamino, N,N-dialkylamino, N,N-diarylamino, or N-aryl-N-(arylalkyl)amino (e.g., N-phenyl-N-(1,1′-biphen-4-ylmethyl)amino); and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates.

In another embodiment, the PDE1 inhibitors for use in the methods of treatment and prophylaxis described herein are Formula 1a:

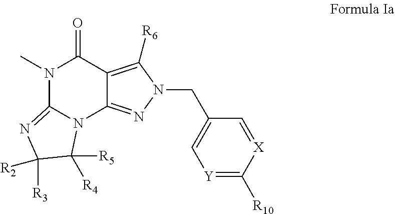

wherein
(i) R₂ and R₅ are independently H or hydroxy and R₃ and R₄ together form a tri- or tetramethylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino; (iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and (iv) X and Y are independently C or N,
in free, pharmaceutically acceptable salt or prodrug form, including its enantiomers, diastereoisomers and racemates.

In another embodiment, the PDE1 inhibitors for use in the methods of treatment and prophylaxis described herein are compounds of Formula II:

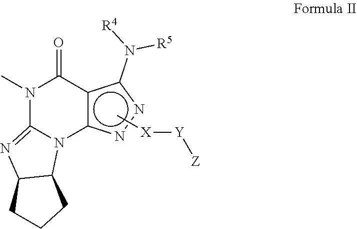

(i) X is C₁₋₆alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), C(O)—R¹, N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹ is C₁₋₆alkyl, haloC₁₋₆alkyl, OH or OC₁₋₆alkyl (e.g., OCH₃);
(v) R² and R³ are independently H or C₁₋₆alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy;
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl), in free, salt or prodrug form.

In yet another embodiment, the PDE1 inhibitors for use in the methods of treatment and prophylaxis described herein are Formula III:

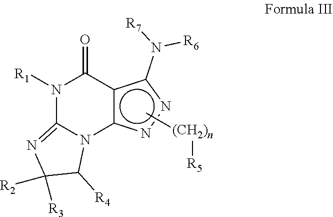

wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,
in free or salt form.

In yet another embodiment, the PDE1 inhibitors for use in the methods of treatment and prophylaxis described herein are Formula IV

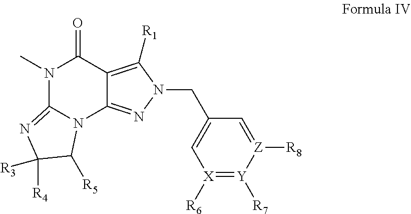

in free or salt form, wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
H,
C₁₋₄alkyl (e.g., methyl),
pyrid-2-yl substituted with hydroxy, or
—S(O)₂—NH₂;
(v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy.

In another embodiment, the PDE1 inhibitors for use in the methods as described herein are Formula V:

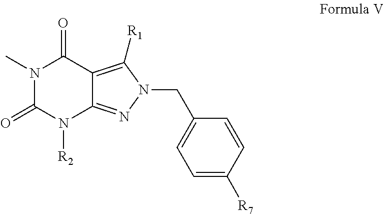

wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl, isobutyl or neopentyl);
(iii) R₃ is —SO₂NH₂ or —COOH;
in free or salt form.

In another embodiment, the PDE1 inhibitors for use in the methods as described herein are Formula VI:

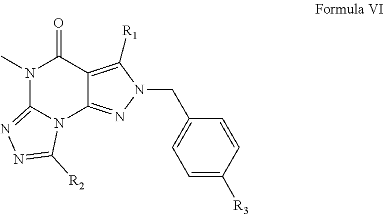

wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl or ethyl);
(iii) R₃ is H, halogen (e.g., bromo), C₁₋₆alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl), in free or salt form.

In one embodiment, the present disclosure provides for administration of a PDE1 inhibitor for use in the methods described herein (e.g., a compound according to Formulas I, Ia, II, III, IV, V, and/or VI), wherein the inhibitor is a compound according to the following:

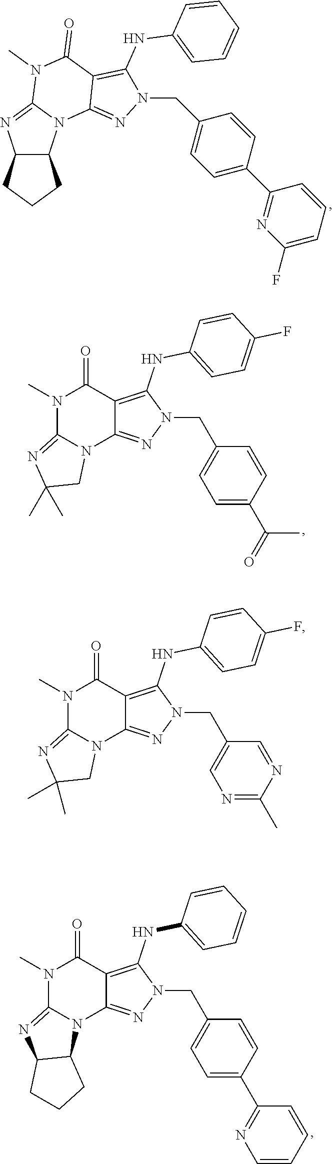

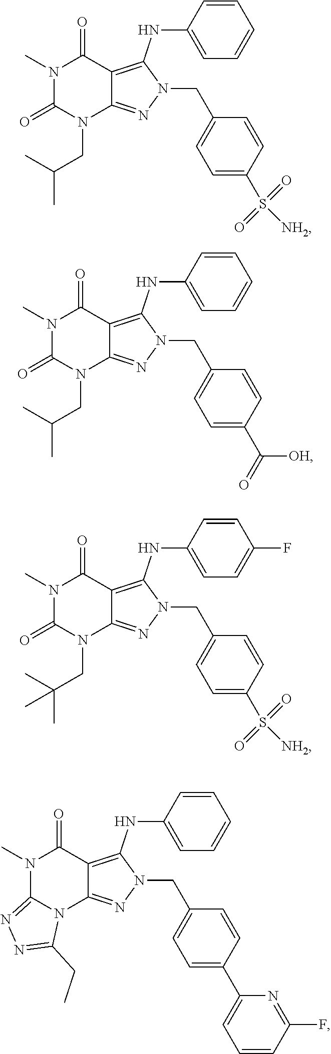

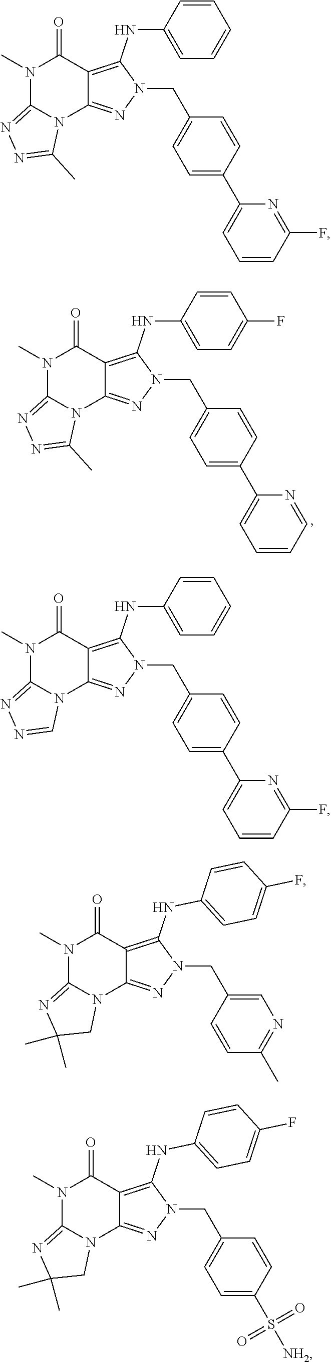

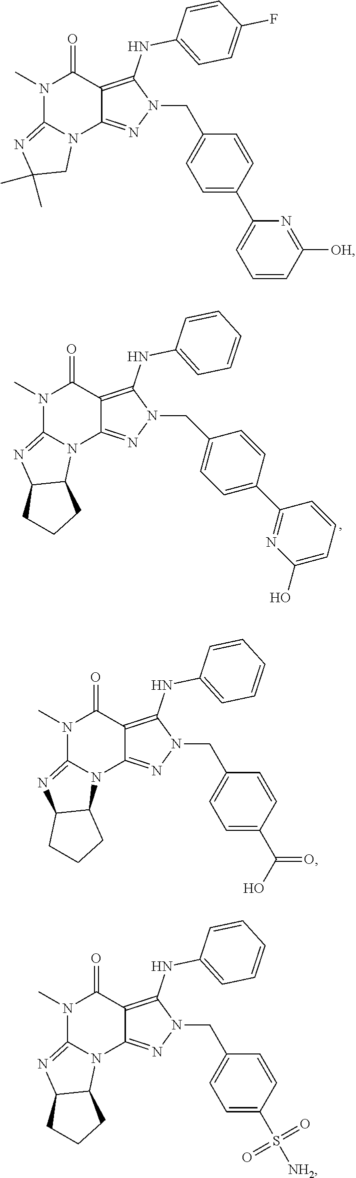

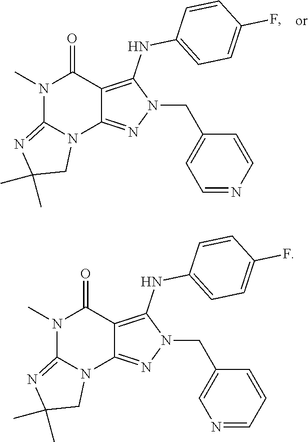

In one embodiment, the invention provides administration of a PDE1 inhibitor for treatment or prophylaxis as described herein, wherein the inhibitor is a compound according to the following:

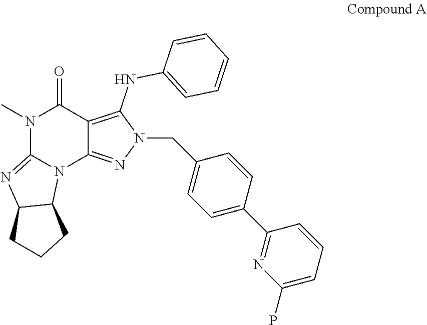

in free or pharmaceutically acceptable salt form.

In still another embodiment, the invention provides administration of a PDE1 inhibitor for treatment or prophylaxis as described herein, wherein the inhibitor is a compound according to the following:

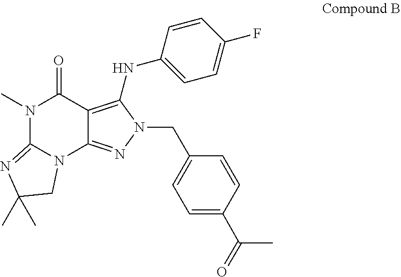 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.

In still another embodiment, the invention provides administration of a PDE1 inhibitor for treatment or prophylaxis as described herein, wherein the inhibitor is a compound according to the following:

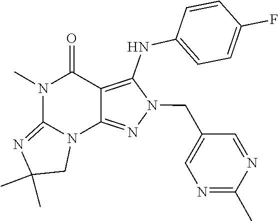 [SMILES: Cc1ncc(Cn2nc3c(c2Nc2ccc(F)cc2)C(=O)N(C)C2=NC(C)(C)CN23)cn1]

in free or pharmaceutically acceptable salt form.

In still another embodiment, the invention provides administration of a PDE1 inhibitor for treatment or prophylaxis as described herein, wherein the inhibitor is a compound according to the following:

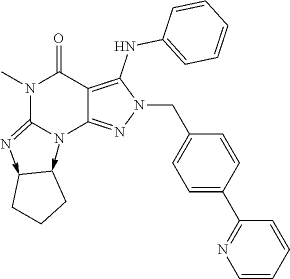

in free or pharmaceutically acceptable salt form.

In one embodiment, selective PDE1 inhibitors of the any of the preceding formulae (e.g., Formulas I, Ia, II, III, IV, V, and/or VI) are compounds that inhibit phosphodiesterase-mediated (e.g., PDE1-mediated, especially PDE1B-mediated) hydrolysis of cGMP, e.g., the preferred compounds have an IC₅₀ of less than 1 μM, preferably less than 500 nM, preferably less than 50 nM, and preferably less than 5 nM in an immobilized-metal affinity particle reagent PDE assay, in free or salt form.

In other embodiments, the invention provides administration of a PDE1 inhibitor for treatment or prophylaxis as described herein, wherein the inhibitor is a compound according to the following:

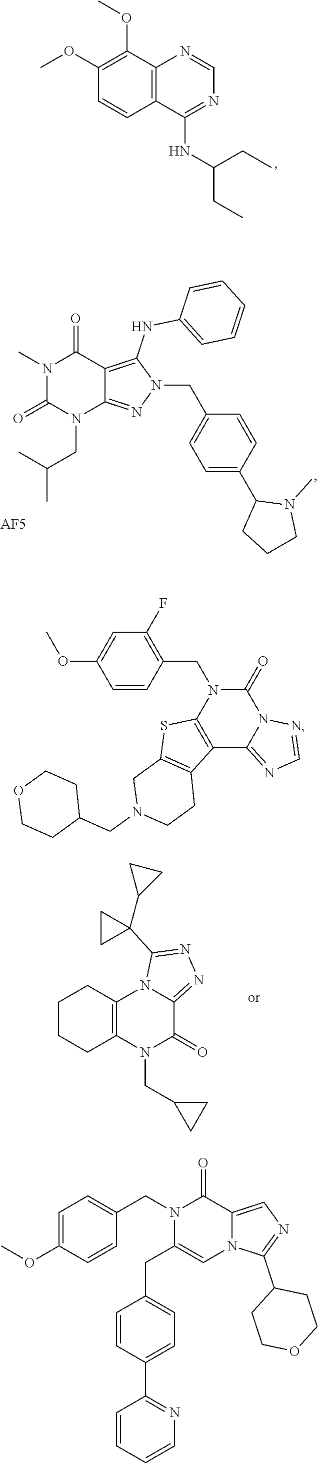

in free or pharmaceutically acceptable salt form.

Further examples of PDE1 inhibitors suitable for use in the methods and treatments discussed herein can be found in International Publication WO2006133261A2; U.S. Pat. Nos. 8,273,750; 9,000,001; 9,624,230; International Publication WO2009075784A1; U.S. Pat. Nos. 8,273,751; 8,829,008; 9,403,836; International Publication WO2014151409A1, U.S. Pat. Nos. 9,073,936; 9,598,426; 9,556,186; U.S. Publication 2017/0231994A1, International Publication WO2016022893A1, and U.S. Publication 2017/0226117A1, each of which are incorporated by reference in their entirety.

Still further examples of PDE1 inhibitors suitable for use in the methods and treatments discussed herein can be found in International Publication WO2018007249A1; U.S. Publication 2018/0000786; International Publication WO2015118097A1; U.S. Pat. No. 9,718,832; International Publication WO2015091805A1; U.S. Pat. No. 9,701,665; U.S. Publication 2015/0175584A1; U.S. Publication 2017/0267664A1; International Publication WO2016055618A1; U.S. Publication 2017/0298072A1; International Publication WO2016170064A1; U.S. Publication 2016/0311831A1; International Publication WO2015150254A1; U.S. Publication 2017/0022186A1; International Publication WO2016174188A1; U.S. Publication 2016/0318939A1; U.S. Publication 2017/0291903A1; International Publication WO2018073251A1; International Publication WO2017178350A1; U.S. Publication 2017/0291901A1; International Publication WO2018/115067; U.S. Publication 2018/0179200A; U.S. Publication US20160318910A1; U.S. Pat. No. 9,868,741; International Publication WO2017/139186A1; International Application WO2016/040083; U.S. Publication 2017/0240532; International Publication WO 2016033776A1; U.S. Publication 2017/0233373; International Publication WO2015130568; International Publication WO2014159012; U.S. Pat. Nos. 9,034,864; 9,266,859; International Publication WO2009085917; U.S. Pat. No. 8,084,261; International Publication WO2018039052; U.S. Publication US20180062729; and International Publication WO2019027783 each of which are incorporated by reference in their entirety. In any situation in which the statements of any documents incorporated by reference contradict or are incompatible with any statements made in the present disclosure, the statements of the present disclosure shall be understood as controlling.

If not otherwise specified or clear from context, the following terms herein have the following meanings:
a. “Selective PDE1 inhibitor” as used herein refers to a PDE1 inhibitor with at least 100-fold selectivity for PDE1 inhibition over inhibition of any other PDE isoform.
b. “Alkyl” as used herein is a saturated or unsaturated hydrocarbon moiety, preferably saturated, preferably having one to six carbon atoms, which may be linear or branched, and may be optionally mono-, di- or tri-substituted, e.g., with halogen (e.g., chloro or fluoro), hydroxy, or carboxy.
c. “Cycloalkyl” as used herein is a saturated or unsaturated nonaromatic hydrocarbon moiety, preferably saturated, preferably comprising three to nine carbon atoms, at least some of which form a nonaromatic mono- or bicyclic, or bridged cyclic structure, and which may be optionally substituted, e.g., with halogen (e.g., chloro or fluoro), hydroxy, or carboxy. Wherein the cycloalkyl optionally contains one or more atoms selected from N and O and/or S, said cycloalkyl may also be a heterocycloalkyl.
d. “Heterocycloalkyl” is, unless otherwise indicated, saturated or unsaturated nonaromatic hydrocarbon moiety, preferably saturated, preferably comprising three to nine carbon atoms, at least some of which form a nonaromatic mono- or bicyclic, or bridged cyclic structure, wherein at least one carbon atom is replaced with N, O or S, which heterocycloalkyl may be optionally substituted, e.g., with halogen (e.g., chloro or fluoro), hydroxy, or carboxy.
e. “Aryl” as used herein is a mono or bicyclic aromatic hydrocarbon, preferably phenyl, optionally substituted, e.g., with alkyl (e.g., methyl), halogen (e.g., chloro or fluoro), haloalkyl (e.g., trifluoromethyl), hydroxy, carboxy, or an additional aryl or heteroaryl (e.g., biphenyl or pyridylphenyl).
f. “Heteroaryl” as used herein is an aromatic moiety wherein one or more of the atoms making up the aromatic ring is sulfur or nitrogen rather than carbon, e.g., pyridyl or thiadiazolyl, which may be optionally substituted, e.g., with alkyl, halogen, haloalkyl, hydroxy or carboxy.

Compounds of the Disclosure, e.g., PDE1 inhibitors as described herein, may exist in free or salt form, e.g., as acid addition salts. In this specification unless otherwise indicated, language such as “Compounds of the Disclosure” is to be understood as embracing the compounds in any form, for example free or acid addition salt form, or where the compounds contain acidic substituents, in base addition salt form. The Compounds of the Disclosure are intended for use as pharmaceuticals, therefore pharmaceutically acceptable salts are preferred. Salts which are unsuitable for pharmaceutical uses may be useful, for example, for the isolation or purification of free Compounds of the Disclosure or their pharmaceutically acceptable salts, are therefore also included.

Compounds of the Disclosure may in some cases also exist in prodrug form. A prodrug form is compound which converts in the body to a Compound of the Disclosure. For example, when the Compounds of the Disclosure contain hydroxy or carboxy substituents, these substituents may form physiologically hydrolysable and acceptable esters. As used herein, “physiologically hydrolysable and acceptable ester” means esters of Compounds of the Disclosure which are hydrolysable under physiological conditions to yield acids (in the case of Compounds of the Disclosure which have hydroxy substituents) or alcohols (in the case of Compounds of the Disclosure which have carboxy substituents) which are themselves physiologically tolerable at doses to be administered. Therefore, wherein the Compound of the Disclosure contains a hydroxy group, for example, Compound-OH, the acyl ester prodrug of such compound, i.e., Compound-O—C(O)—C1-4alkyl, can hydrolyze in the body to form physiologically hydrolysable alcohol (Compound-OH) on the one hand and acid on the other (e.g., HOC(O)—C1-4alkyl). Alternatively, wherein the Compound of the Disclosure contains a carboxylic acid, for example, Compound-C(O)OH, the acid ester prodrug of such compound, Compound-C(O)O—C1-4alkyl can hydrolyze to form Compound-C(O)OH and HO—C1-4alkyl. As will be appreciated the term thus embraces conventional pharmaceutical prodrug forms.

In another embodiment, the disclosure further provides a pharmaceutical composition comprising a PDE1 inhibitor in combination with an immune checkpoint inhibitor, each in free or pharmaceutically acceptable salt form, in admixture with a pharmaceutically acceptable carrier. The term “combination,” as used herein, embraces simultaneous, sequential, or contemporaneous administration of the PDE1 inhibitor and the immune checkpoint inhibitor. In some embodiments, the combination of the PDE1 inhibitor and the immune checkpoint inhibitor allows the immune checkpoint inhibitor to be administered in a dosage lower than would be effective if administered as sole monotherapy.

### Methods of Using Compounds of the Disclosure

In one embodiment, the present application provides a method (Method 1) of treating breast cancer comprising administering a pharmaceutically acceptable amount of a PDE1 inhibitor (e.g., PDE1 inhibitor according to Formulas I, Ia, II, III, IV, V, and/or VI) alone or in combination with a pharmaceutically acceptable amount of an immune checkpoint inhibitor to a subject in need thereof.
1.1 Method 1, wherein the breast cancer has elevated expression of PDE1.
1.2 Any preceding method, wherein the breast cancer expresses PD-L1, e.g., Combined Positive Score (CPS)≥1 as determined by an FDA approved test, wherein CPS is the number of PD-L1 staining cells (tumor cells, lymphocytes, macrophages) divided by the total number of viable tumor cells, multiplied by 100.
1.3 Any preceding method, wherein the breast cancer is triple-negative breast cancer (TNBC), which is estrogen receptor-negative, progesterone receptor-negative, and HER2-negative.
1.4 Method 1.3, wherein the TNBC is a high-risk early stage TNBC.
1.5 Method 1.3, wherein the treatment is an adjuvant treatment after surgery.
1.6 Method 1.3, wherein the subject has locally recurrent unresectable or metastatic TNBC whose tumors express PD-L1, e.g., Combined Positive Score (CPS)≥1 as determined by an FDA approved test, wherein CPS is the number of PD-L1 staining cells (tumor cells, lymphocytes, macrophages) divided by the total number of viable tumor cells, multiplied by 100.
1.7 Any preceding method, wherein the administration of the pharmaceutically acceptable amount of the PDE1 inhibitor in combination with the pharmaceutically acceptable amount of the immune checkpoint inhibitor to the subject increases the M1/M2 ratio of macrophages in the tumor microenvironment.
1.8 Any preceding method, wherein administering the pharmaceutically acceptable amount of the immune checkpoint inhibitor alone (i.e., not in combination with the PDE1 inhibitor) to the subject is not effective in treating the breast cancer, e.g., it does not inhibit the growth of the breast cancer.
1.9 Any preceding method, wherein administering the pharmaceutically acceptable amount of the PDE1 inhibitor alone (i.e., not in combination with the checkpoint inhibitor) to the subject is not effective in treating the breast cancer, e.g., it does not inhibit the growth of the breast cancer.
1.10 Any preceding method, wherein the immune checkpoint inhibitor is selected from one or more of inhibitors of CTLA-4, PD-1 and/or PD-L1.
1.11 Any preceding method, wherein the immune checkpoint inhibitor is an inhibitor of PD-1, optionally wherein the immune checkpoint inhibitor is anti-PD-1 antibody, further optionally wherein the antibody is monoclonal or polyclonal.
1.12 Any preceding method, wherein the immune checkpoint inhibitor is an inhibitor of PD-L1, optionally wherein the immune checkpoint inhibitor is anti-PD-L1 antibody, further optionally wherein the antibody is monoclonal or polyclonal.
1.13 Any preceding method, wherein the immune checkpoint inhibitor is an inhibitor of CTLA-4, optionally wherein the immune checkpoint inhibitor is anti-CTLA-4 antibody, further optionally wherein the antibody is monoclonal or polyclonal.
1.14 Any preceding method, wherein the immune checkpoint inhibitor comprises one or more members selected from nivolumab, pembrolizumab, cemiplimab, ipilimumab, avelumab, durvalumab, atezolizumab, and spartalizumab.
1.15 Any preceding method, wherein the subject is suffering from a systemic inflammatory response, a gastrointestinal inflammation-related disorder, an endocrine inflammation-related disorder, a dermatologic inflammation-related disorder, an ophthalmologic inflammation-related disorder, a neurologic inflammation-related disorder, a hematologic inflammation-related disorder, a genitourinary inflammation-related disorder, a respiratory inflammation-related disorder, a musculoskeletal inflammation-related disorder, a cardiac inflammation-related disorder, or a defined systemic inflammation-related disorder.
1.16 Any of the preceding methods, wherein the subject in need thereof was previously administered an immune checkpoint inhibitor therapy, optionally wherein the pharmaceutically acceptable amount of the immune checkpoint inhibitor administered to the subject is lower than the amount of the immune checkpoint inhibitor administered in the previous immune checkpoint inhibitor therapy.
1.17 Any of the preceding methods, wherein the subject in need thereof is suffering from a disease, disorder or adverse effect of immune checkpoint inhibitor therapy.
1.18 Any preceding method, wherein the subject is suffering from an inflammatory-related disorder consequent to immune checkpoint inhibitor therapy.
1.19 Any preceding method wherein the subject is suffering from, or at risk of suffering from, a side effect of immune checkpoint inhibitor therapy, e.g., wherein the side effect is selected from a systemic inflammatory response, a gastrointestinal inflammation-related disorder, an endocrine inflammation-related disorder, a dermatologic inflammation-related disorder, an ophthalmologic inflammation-related disorder, a neurologic inflammation-related disorder, a hematologic inflammation-related disorder, a genitourinary inflammation-related disorder, a respiratory inflammation-related disorder, a musculoskeletal inflammation-related disorder, a cardiac inflammation-related disorder, or a defined systemic inflammation-related disorder.
1.20 Any preceding method wherein the subject is suffering from a side effect of immune checkpoint inhibitor therapy, wherein the side effect is a gastrointestinal inflammation-related disorder, e.g., selected from colitis, enterocolitis, colitis complicated by intestinal perforation, hepatitis, and pancreatitis.
1.21 Any preceding method wherein the subject is suffering from a side effect of immune checkpoint inhibitor therapy, wherein the side effect is endocrine inflammation-related disorder, e.g., selected from hypophysitis (e.g., manifested as panhypopituitarism), thyrotoxicosis, hypothyroidism, syndrome of inappropriate secretion of antidiuretic hormone, central adrenal insufficiency, primary adrenal insufficiency, and diabetes mellitus.
1.22 Any preceding method wherein the subject is suffering from a side effect of immune checkpoint inhibitor therapy, wherein the side effect is a dermatologic inflammation-related disorder, e.g., selected from a rash, pruritis, vitiligo, dermatitis, sweet syndrome, drug eruption, poliosis, delayed hypersensitivity reaction, alopecia universalis, grover's disease, pyoderma gangrenosum, toxic epidermal necrolysis, chronic non-caseation granuloma, bullous pemphigoid, and psoriasis.
1.23 Any preceding method wherein the subject is suffering from a side effect of immune checkpoint inhibitor therapy, wherein the side effect is an ophthalmologic inflammation-related disorder, e.g., selected from uveitis, conjunctivitis, orbital inflammation, Grave's ophthalmology, choroidal neovascularization, optic neuropathy, keratitis, and retinopathy.
1.24 Any preceding method wherein the subject is suffering from a side effect of immune checkpoint inhibitor therapy, wherein the side effect is a neurologic inflammation-related disorder, e.g., selected from encephalopathy, Guillan-Barre syndrome, polyradiculoneuropathy, symmetrical multifocal neuropathy, transverse myelitis, necrotizing myelopathy, myasthenia gravis, phrenic nerve palsy, immune related meningitis, meningoradiculoneuritis, peripheral neuropathy, autoimmune inner ear disease, multiple sclerosis, and inflammatory enteric neuropathy.
1.25 Any preceding method wherein the subject is suffering from a side effect of immune checkpoint inhibitor therapy, wherein the side effect is a hematologic inflammation-related disorder, e.g., selected from thrombocytopenia, pancytopenia, neutropenia, eosinophilia, pure red blood cell aplasia, acquired hemophilia A, and disseminated intravascular coagulopathy.
1.26 Any preceding method wherein the subject is suffering from a side effect of immune checkpoint inhibitor therapy, wherein the side effect is a genitourinary inflammation-related disorder, e.g., selected from renal failure, acute/granulomatous interstitial nephritis, acute tubular necrosis, and lymphotic vasculitis (e.g., lymphotic vasculitis of the uterus).
1.27 Any preceding method wherein the subject is suffering from a side effect of immune checkpoint inhibitor therapy, wherein the side effect is a respiratory inflammation-related disorder, e.g., selected from pneumonitis and acute respiratory distress.
1.28 Any preceding method wherein the subject is suffering from a side effect of immune checkpoint inhibitor therapy, wherein the side effect is a musculoskeletal inflammation-related disorder, e.g., selected from polyarthritis, athralgia, myalgia, chronic granulomatous inflammation of rectus abdominis muscle, and rhabdomyolysis.
1.29 Any preceding method wherein the subject is suffering from a side effect of immune checkpoint inhibitor therapy, wherein the side effect is a cardiac inflammation-related disorder, e.g., selected from precarditis and takotsubo like syndrome.
1.30 Any preceding method wherein the subject is suffering from a side effect of immune checkpoint inhibitor therapy, wherein the side effect is a systemic inflammation-related disorder, e.g., selected from lung sarcoidosis, cutaneous and pulmonary sarcoidosis, polymyalgia rheumatica, giant cell arteritis, muscular sarcoidosis, neurological and pulmonary sarcoidosis, celiac disease, lupus nephritis, dermatomyositis, autoimmune inflammatory myopathy, and Vogt-Koyanagi like syndrome.
1.31 Any of the preceding methods, wherein the PDE1 inhibitor and the immune checkpoint inhibitor are administered before, after or in conjunction with radiation therapy or chemotherapy.
1.32 Any of the preceding methods, wherein the PDE1 inhibitor and the immune checkpoint inhibitor are administered concurrently with radiation therapy or chemotherapy.
1.33 Any of the preceding methods, wherein the PDE1 inhibitor and the immune checkpoint inhibitor are administered prior to radiation therapy or chemotherapy.
1.34 Any of the preceding methods, wherein the PDE1 inhibitor and the immune checkpoint inhibitor are administered after radiation therapy or chemotherapy.
1.35 Any of the preceding methods, wherein the PDE1 inhibitor and the immune checkpoint inhibitor are administered together with an additional antitumor agent, chemotherapeutic, gene therapeutic and/or immunologic treatment.
1.36 Any of the preceding methods, wherein the PDE1 inhibitor is a PDE1 inhibitor according to Formulas I, Ia, II, III, IV, V, and/or VI or a compound according to the following:

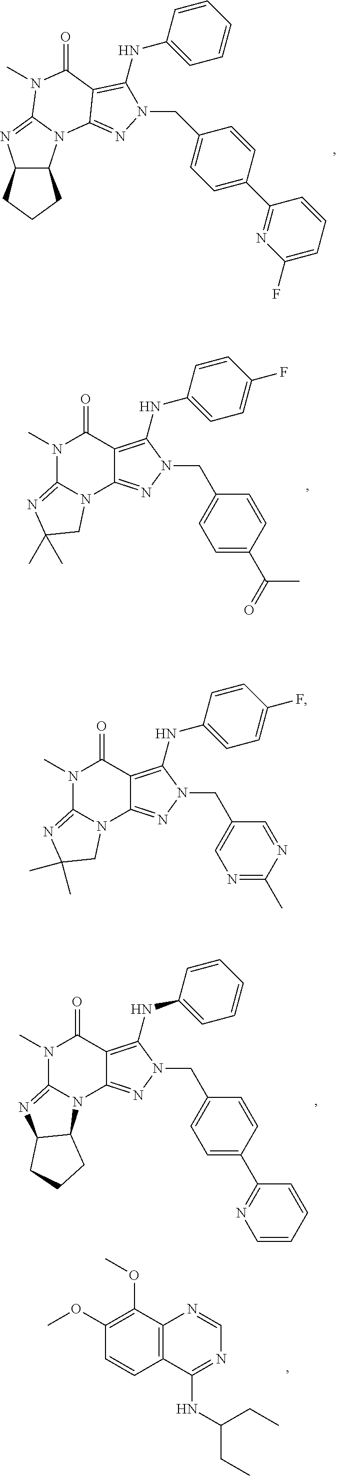

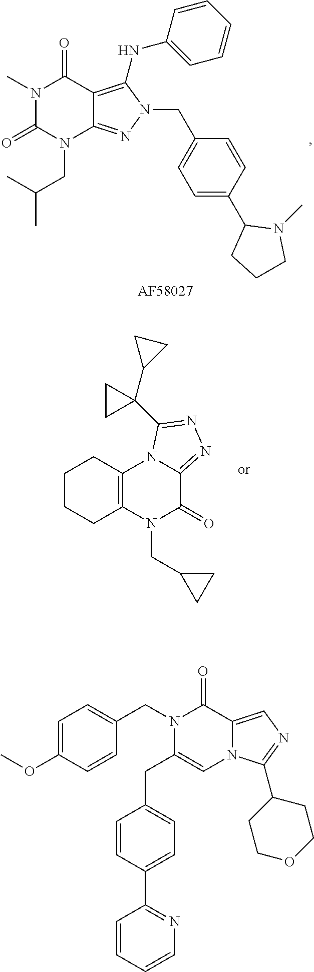

in free or pharmaceutically acceptable salt form.
1.37 Any of the preceding methods, wherein the PDE1 inhibitor is a PDE1 inhibitor according to Formula Ia.
1.38 Any of the preceding methods, wherein the PDE1 inhibitor is a compound as follows:

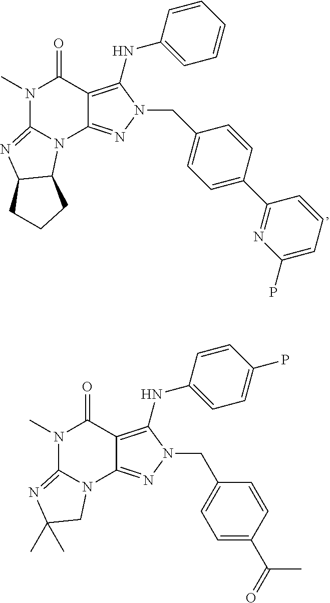

in free or pharmaceutically acceptable salt form.
1.39 Any of the preceding methods, wherein the PDE1 inhibitor is a compound as follows:

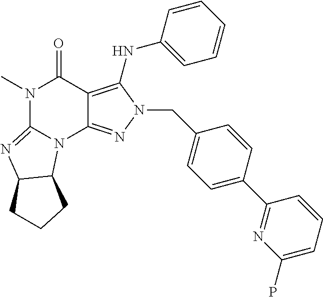

in free or pharmaceutically acceptable salt form, e.g., in monophosphate salt form.
1.40 Any of the preceding methods, further comprising administering a pharmaceutically acceptable amount of a beta blocker to the subject.
1.41 Any preceding methods, wherein the subject is human.

In another embodiment, the disclosure provides a PDE1 inhibitor alone or in combination with an immune checkpoint inhibitor for use to treat breast cancer, e.g., for use in any of Methods 1, et seq.

In another embodiment, the present application provides a method (Method 2) of prophylaxis or mitigation of a disease, disorder or adverse effect consequent to administration of an immune checkpoint inhibitor to a subject suffering from breast cancer, the method comprising reducing the amount of checkpoint inhibitor administered to the subject and administering a pharmaceutically acceptable amount of a PDE1 inhibitor (i.e., PDE1 inhibitor according to Formulas I, Ia, II, III, IV, V, and/or VI) in combination with the immune checkpoint inhibitor therapy to the subject.
2.1 Method 2, wherein the breast cancer has elevated expression of PDE1.
2.2 Any preceding method, wherein the breast cancer expresses PD-L1, e.g., Combined Positive Score (CPS)≥1 as determined by an FDA approved test, wherein CPS is the number of PD-L1 staining cells (tumor cells, lymphocytes, macrophages) divided by the total number of viable tumor cells, multiplied by 100.
2.3 Any preceding method, wherein the breast cancer is triple-negative breast cancer (TNBC).
2.4 Method 2.3, wherein the TNBC is a high-risk early stage TNBC.
2.5 Method 2.3, wherein the immune checkpoint inhibitor therapy is an adjuvant treatment after surgery.
2.6 Method 2.3, wherein the subject has locally recurrent unresectable or metastatic TNBC whose tumors express PD-L1, e.g., Combined Positive Score (CPS) 1 as determined by an FDA approved test, wherein CPS is the number of PD-L1 staining cells (tumor cells, lymphocytes, macrophages) divided by the total number of viable tumor cells, multiplied by 100.
2.7 Any preceding method, wherein the reduced amount of the immune checkpoint inhibitor when administered alone (i.e., not in combination with the PDE1 inhibitor) to the subject is not effective in treating the breast cancer, e.g., administering the reduced amount of the immune checkpoint inhibitor alone (i.e., not in combination with the PDE1 inhibitor) to the subject does not inhibit the growth of the breast cancer.
2.8 Any preceding method, wherein the administration of the pharmaceutically acceptable amount of the PDE1 inhibitor in combination with the reduced amount of the immune checkpoint inhibitor to the subject increases the M1/M2 ratio of macrophages in the tumor microenvironment.
2.9 Any preceding method, wherein the immune checkpoint inhibitor is selected from one or more of inhibitors of CTLA-4, PD-1 and/or PD-L1.
2.10 Any preceding method, wherein the immune checkpoint inhibitor is an inhibitor of PD-1, optionally wherein the immune checkpoint inhibitor is anti-PD-1 antibody, further optionally wherein the antibody is monoclonal or polyclonal.
2.11 Any preceding method, wherein the immune checkpoint inhibitor is an inhibitor of PD-L1, optionally wherein the immune checkpoint inhibitor is anti-PD-L1 antibody, further optionally wherein the antibody is monoclonal or polyclonal.
2.12 Any preceding method, wherein the immune checkpoint inhibitor is an inhibitor of CTLA-4, optionally wherein the immune checkpoint inhibitor is anti-CTLA-4 antibody, further optionally wherein the antibody is monoclonal or polyclonal.
2.13 Any preceding method, wherein the immune checkpoint inhibitor comprises one or more members selected from nivolumab, pembrolizumab, cemiplimab, ipilimumab, avelumab, durvalumab, atezolizumab, and spartalizumab.
2.14 Any preceding method, wherein the subject is suffering from an inflammatory-related disorder consequent to the immune checkpoint inhibitor therapy.
2.15 Any preceding method, wherein the subject is suffering from a systemic inflammatory response, a gastrointestinal inflammation-related disorder, an endocrine inflammation-related disorder, a dermatologic inflammation-related disorder, an ophthalmologic inflammation-related disorder, a neurologic inflammation-related disorder, a hematologic inflammation-related disorder, a genitourinary inflammation-related disorder, a respiratory inflammation-related disorder, a musculoskeletal inflammation-related disorder, a cardiac inflammation-related disorder, or a defined systemic inflammation-related disorder.
2.16 Method 2.15, wherein the gastrointestinal inflammation-related disorder is selected from colitis, enterocolitis, colitis complicated by intestinal perforation, hepatitis, and pancreatitis.
2.17 Method 2.15, wherein the endocrine inflammation-related disorder is selected from hypophysitis (e.g., manifested as panhypopituitarism), thyrotoxicosis, hypothyroidism, syndrome of inappropriate secretion of antidiuretic hormone, central adrenal insufficiency, primary adrenal insufficiency, and diabetes mellitus.
2.18 Method 2.15, wherein the dermatologic inflammation-related disorder is selected from a rash, pruritis, vitiligo, dermatitis, sweet syndrome, drug eruption, poliosis, delayed hypersensitivity reaction, alopecia universalis, grover's disease, pyoderma gangrenosum, toxic epidermal necrolysis, chronic non-caseation granuloma, bullous pemphigoid, and psoriasis.
2.19 Method 2.15, wherein the ophthalmologic inflammation-related disorder is selected from uveitis, conjunctivitis, orbital inflammation, Grave's ophthalmology, choroidal neovascularization, optic neuropathy, keratitis, and retinopathy.
2.20 Method 2.15, wherein the neurologic inflammation-related disorder is selected from encephalopathy, Guillan-Barre syndrome, polyradiculoneuropathy, symmetrical multifocal neuropathy, transverse myelitis, necrotizing myelopathy, myasthenia gravis, phrenic nerve palsy, immune related meningitis, meningoradiculoneuritis, peripheral neuropathy, autoimmune inner ear disease, multiple sclerosis, and inflammatory enteric neuropathy.
2.21 Method 2.15, wherein the hematologic inflammation-related disorder is selected from thrombocytopenia, pancytopenia, neutropenia, eosinophilia, pure red blood cell aplasia, acquired hemophilia A, and disseminated intravascular coagulopathy.
2.22 Method 2.15, wherein the genitourinary inflammation-related disorder is selected from renal failure, acute/granulomatous interstitial nephritis, acute tubular necrosis, and lymphotic vasculitis (e.g., lymphotic vasculitis of the uterus).
2.23 Method 2.15, wherein the respiratory inflammation-related disorder is selected from pneumonitis and acute respiratory distress.
2.24 Method 2.15, wherein the musculoskeletal inflammation-related disorder is selected from polyarthritis, athralgia, myalgia, chronic granulomatous inflammation of rectus abdominis muscle, and rhabdomyolysis.
2.25 Method 2.15, wherein the cardiac inflammation-related disorder is selected from precarditis and takotsubo like syndrome.
2.26 Method 2.15, wherein the defined systemic inflammation-related disorder is selected from lung sarcoidosis, cutaneous and pulmonary sarcoidosis, polymyalgia rheumatica, giant cell arteritis, muscular sarcoidosis, neurological and pulmonary sarcoidosis, celiac disease, lupus nephritis, dermatomyositis, autoimmune inflammatory myopathy, and Vogt-Koyanagi like syndrome.
2.27 Any of the preceding methods, wherein the PDE1 inhibitor and the immune checkpoint inhibitor are administered with radiation therapy or chemotherapy.
2.28 Any of the preceding methods, wherein the PDE1 inhibitor and the immune checkpoint inhibitor are administered concurrently with radiation therapy or chemotherapy.
2.29 Any of the preceding methods, wherein the PDE1 inhibitor and the immune checkpoint inhibitor are administered prior to radiation therapy or chemotherapy.
2.30 Any of the preceding methods, wherein the PDE1 inhibitor and the immune checkpoint inhibitor are administered after radiation therapy or chemotherapy.
2.31 Any of the preceding methods, wherein the PDE1 inhibitor and the immune checkpoint inhibitor are administered together with an additional antitumor agent, chemotherapeutic, gene therapeutic and/or immunologic treatment.
2.32 Any of the preceding methods, wherein the PDE1 inhibitor is a PDE1 inhibitor according to Formulas I, Ia, II, III, IV, V, and/or VI or a compound according to the following:

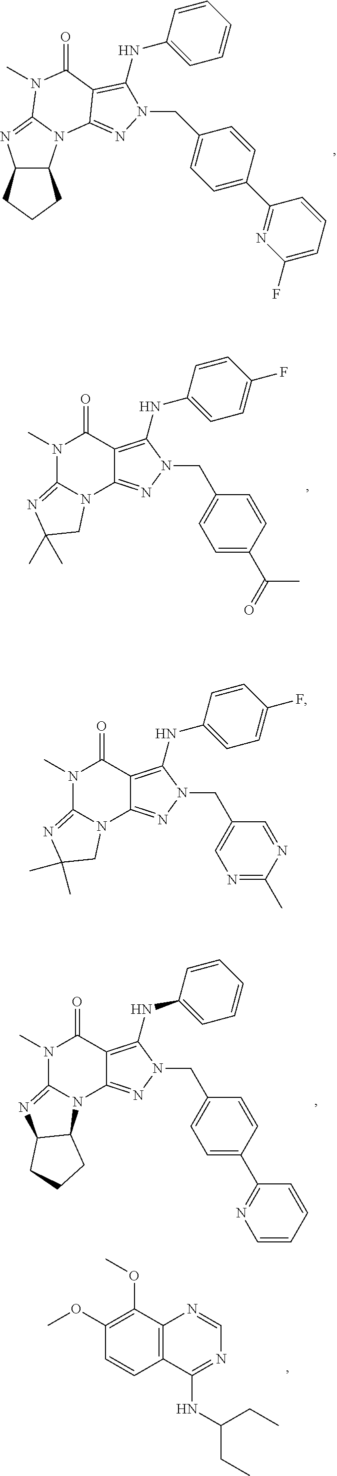

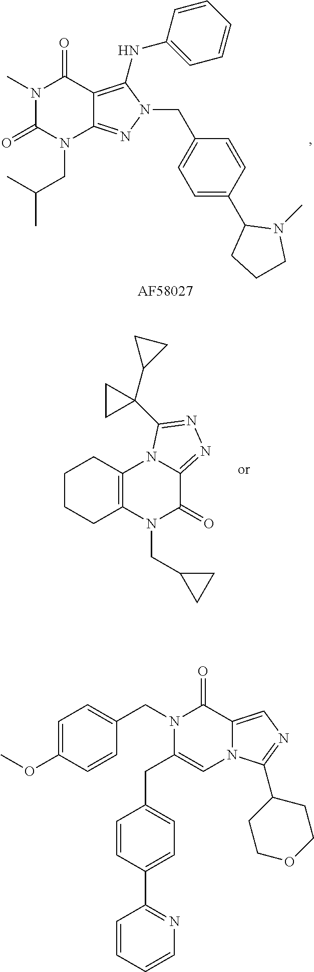

in free or pharmaceutically acceptable salt form.
2.33 Any of the preceding methods, wherein the PDE1 inhibitor is a PDE1 inhibitor according to Formula Ia.
2.34 Any of the preceding methods, wherein the PDE1 inhibitor is a compound as follows:

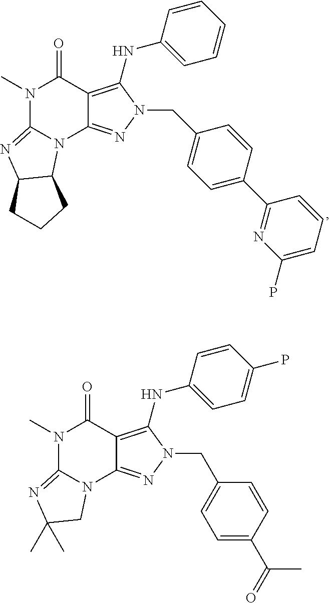

in free or pharmaceutically acceptable salt form.
2.35 Any of the preceding methods, wherein the PDE1 inhibitor is a compound as follows:

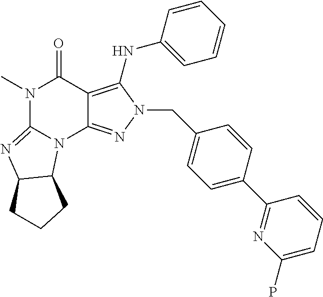

in free or pharmaceutically acceptable salt form e.g., monophosphate salt form.
2.36 Any of the preceding methods, further comprising administering a pharmaceutically acceptable amount of a beta blocker to the subject.
2.37 Any of the preceding methods, wherein the subject is human.

In another embodiment, the disclosure provides a PDE1 inhibitor (e.g., a PDE1 inhibitor according to Formulas I, Ia, II, III, IV, V, and/or VI) for use in prophylaxis or mitigation of a disease, disorder or adverse effect consequent to administration of a checkpoint inhibitor therapy, e.g., for use in any of methods 2, et seq.

Combination Therapies with PDE1 Inhibitors

In the present invention, the PDE1 inhibitor may be administered in combination with an immune checkpoint inhibitor. The combination therapy may be achieved by administering a single composition or pharmacological formulation that includes the PDE1 inhibitor and the immune checkpoint inhibitor, or by administration of two distinct compositions or formulations, separately, simultaneously or sequentially, wherein one composition includes the PDE1 inhibitor and the other includes the immune checkpoint inhibitor. The therapy using an inhibitor may precede or follow administration of the other inhibitor by intervals ranging from minutes to weeks. In embodiments where the other inhibitor is applied separately to the cell, one would generally ensure that a significant period of time did not expire between the time of each delivery, such that the PDE1 inhibitor and the immune checkpoint inhibitor would still be able to exert an advantageously combined effect on the cell. In some embodiments, it is contemplated that one would typically contact the cell with both inhibitors within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It is also conceivable that more than one administration of either a PDE1 inhibitor, or an immune checkpoint inhibitor will be desired. In this regard, various combinations may be employed. By way of illustration, where the PDE1 inhibitor is “A” and the immune checkpoint inhibitor is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Accordingly, in various embodiments, the present disclosure also provides a pharmaceutical combination [Combination 1] therapy comprising a pharmaceutically acceptable amount of a PDE1 inhibitor (e.g., a compound according to Formulas I, Ia, II, III, IV, V, and/or VI) and a pharmaceutically acceptable amount of an immune checkpoint inhibitor, for administration in a method of treating breast cancer, e.g., in accordance with any of Method 1, et seq., or for prophylaxis or mitigation of a disease, disorder or adverse effect consequent to administration of a checkpoint inhibitor therapy, e.g. in accordance with any of Method 2, et seq. For example, the present disclosure provides the following Combinations:
1.1 Combination 1, wherein the PDE1 inhibitor and the checkpoint inhibitor are in a single dosage form, e.g., a tablet or capsule, in combination or association with a pharmaceutically acceptable diluent or carrier.
1.2 Combination 1, wherein the PDE1 inhibitor and the checkpoint inhibitor are in a single package, e.g., with instructions for administration simultaneously or sequentially.
1.3 Any of the preceding combinations, wherein the PDE1 inhibitor is a PDE1 inhibitor according to Formulas I, Ia, II, III, IV, V, and/or VI or a compound according to the following:

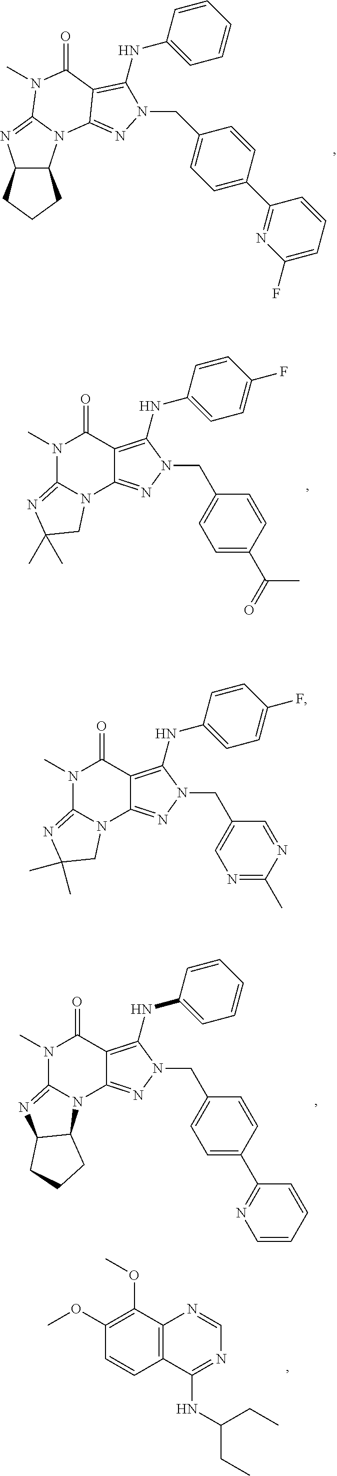

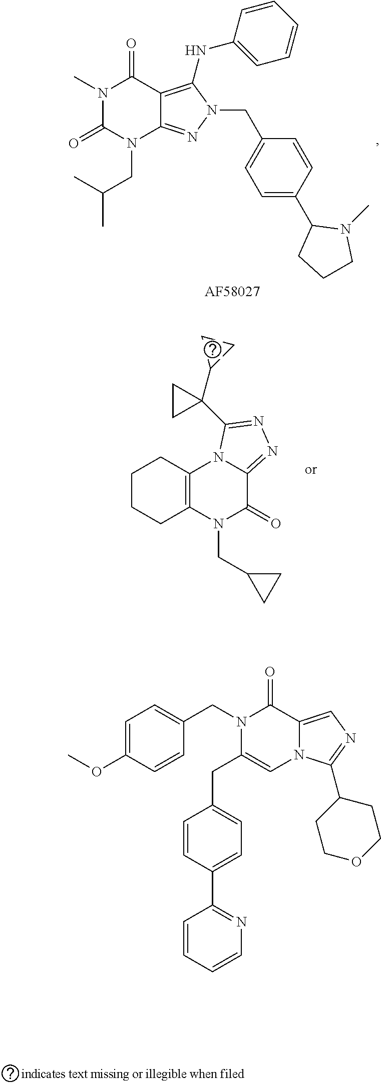

in free or pharmaceutically acceptable salt form.
1.4 Any of the preceding combinations, wherein the PDE1 inhibitor is a PDE1 inhibitor according to Formula Ia.
1.5 Any of the preceding combinations, wherein the PDE1 inhibitor is a compound as follows:

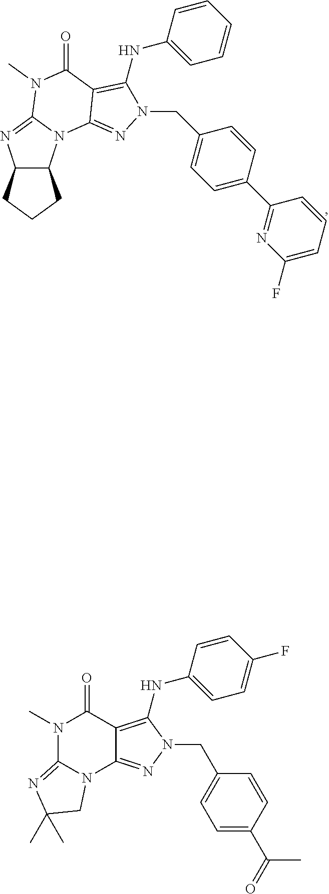 [SMILES: CN1C(=O)c2c(nn(Cc3ccc(-c4cccc(F)n4)cc3)c2Nc2ccccc2)N2C1=N[C@@H]1CCC[C@@H]12 ; CC(=O)c1ccc(Cn2nc3c(c2Nc2ccc(F)cc2)C(=O)N(C)C2=NC(C)(C)CN23)cc1]

in free or pharmaceutically acceptable salt form.
1.6 Any of the preceding combinations, wherein the PDE1 inhibitor is a compound as follows:

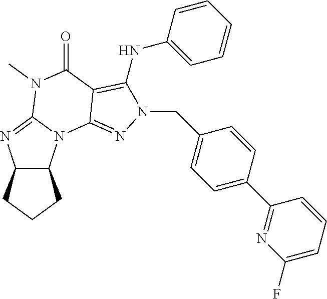 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.7 Any of the preceding combinations, wherein the immune checkpoint inhibitor is selected from one or more of inhibitors of CTLA-4, PD-1 and/or PD-L1.
1.8 Any of the preceding combinations, wherein the immune checkpoint inhibitor is an inhibitor of PD-1, optionally wherein the immune checkpoint inhibitor is anti-PD-1 antibody, further optionally wherein the antibody is monoclonal or polyclonal.
1.9 Any of the preceding combinations, wherein the immune checkpoint inhibitor is an inhibitor of PD-L1, optionally wherein the immune checkpoint inhibitor is anti-PD-L1 antibody, further optionally wherein the antibody is monoclonal or polyclonal.
1.10 Any of the preceding combinations, wherein the immune checkpoint inhibitor is an inhibitor of CTLA-4, optionally wherein the immune checkpoint inhibitor is anti-CTLA-4 antibody, further optionally wherein the antibody is monoclonal or polyclonal.
1.11 Any of the preceding combinations, wherein the immune checkpoint inhibitor comprises one or more members selected from nivolumab, pembrolizumab, cemiplimab, ipilimumab, avelumab, durvalumab, atezolizumab, and spartalizumab.
1.12 Any of the preceding combinations, wherein the checkpoint inhibitor is administered concurrently with the PDE1 inhibitor.
1.13 Any of the preceding combinations, wherein the checkpoint inhibitor is administered prior to administering the PDE1 inhibitor.
1.14 Any of the preceding combinations, wherein the checkpoint inhibitor is administered after administering the PDE1 inhibitor.
1.15 Any of the preceding combinations, wherein the subject in need thereof was previously or concurrently administered a checkpoint inhibitor therapy.
1.16 Any of the preceding combinations, further comprising a pharmaceutically acceptable amount of a beta blocker.

In some embodiments, the pharmaceutical compositions are administered in combination with one or more additional antitumor drugs, for example, drugs known to have an effect in treating or eliminating cancers and/or tumors. Non-limiting examples of antitumor drugs are Abemaciclib, Abiraterone Acetate, Abitrexate (Methotrexate), Abraxane (Paclitaxel Albumin-stabilized Nanoparticle Formulation), ABVD, ABVE, ABVE-PC, AC, Acalabrutinib, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia, Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Ciloleucel, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calquence (Acalabrutinib), Campath (Alemtuzumab), Camptosar (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil-Topical, Flutamide, Folex (Methotrexate), Folex PFS (Methotrexate), Folfiri, Folfiri-Bevacizumab, Folfiri-Cetuximab, Folfirinox, Folfox, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspagase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R—CHOP, R—CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate).

In some embodiments, the PDE1 inhibitor and the immune checkpoint inhibitor are administered in combination with one or more beta blockers. A non-exhaustive list of such beta blockers includes various beta-adrenergic receptor antagonists, also called beta-blockers, are currently in clinical use for eliminating the harmful chronic myocardial stimulation which is caused by failing heart. Preferred beta-adrenergic receptor antagonists include metoprolol, metoprolol succinate, carvedilol, atenolol, propranolol, acebutolol, acebutolol HCL, betaxolol, betaxolol HCL, nadolol, talinolol, bisoprolol, bisoprolol hemifumarate, carteolol, carteolol HCL, esmolol, esmolol HCL, labetalol, labetalol HCL, metoprolol, metoprolol succinate, metoprolol tartrate, nadolol, penbutolol, penbutolol sulfate, pindolol, propranolol, propranolol HCL, sotalol, sotalol HCL, timolol and timolol hydrogen maleate salt or a pharmaceutically acceptable salt thereof. According to the invention, a beta-adrenergic receptor antagonist may be administered in daily doses, which are clinically accepted for such agents. For example, a suitable daily dose of metoprolol as a tartrate or succinate salt, is about 100-200 mg and for carvedilol about 5-50 mg depending upon the condition to be treated, the route of administration, age, weight and the condition of the patient.

As used herein, the term “antitumor agent” is understood to refer to any chemical agents or drugs effective in preventing or inhibiting the formation or growth of cancers or tumors. Antitumor agents as discussed herein may encompass alkylating agents, antimetabolites, natural products, hormones, and/or antibodies. Treatment of tumors or cancer may include limiting the proliferation, migration and/or invasion of cancerous or tumorous cells in the body, or limiting the symptoms associated with said cancer or tumor. As used herein, antitumor agents are understood to encompass and otherwise be synonymous with anticancer agents.

### Methods of Making Compounds of the Disclosure

The PDE1 inhibitors of the Disclosure and their pharmaceutically acceptable salts may be made using the methods as described and exemplified in U.S. Pat. No. 8,273,750, US 2006/0173878, U.S. Pat. No. 8,273,751, US 2010/0273753, U.S. Pat. Nos. 8,697,710, 8,664,207, 8,633,180, 8,536,159, US 2012/0136013, US 2011/0281832, US 2013/0085123, US 2013/0324565, US 2013/0338124, US 2013/0331363, WO 2012/171016, and WO 2013/192556, and by methods similar thereto and by methods known in the chemical art. Such methods include, but not limited to, those described below. If not commercially available, starting materials for these processes may be made by procedures, which are selected from the chemical art using techniques which are similar or analogous to the synthesis of known compounds.

Various PDE1 inhibitors and starting materials therefor may be prepared using methods described in US 2008-0188492 A1, US 2010-0173878 A1, US 2010-0273754 A1, US 2010-0273753 A1, WO 2010/065153, WO 2010/065151, WO 2010/065151, WO 2010/065149, WO 2010/065147, WO 2010/065152, WO 2011/153129, WO 2011/133224, WO 2011/153135, WO 2011/153136, WO 2011/153138. All references cited herein are hereby incorporated by reference in their entirety.

The Compounds of the Disclosure (PDE1 inhibitors) include their enantiomers, diastereomers and racemates, as well as their polymorphs, hydrates, solvates and complexes. Some individual compounds within the scope of this disclosure may contain double bonds. Representations of double bonds in this disclosure are meant to include both the E and the Z isomer of the double bond. In addition, some compounds within the scope of this disclosure may contain one or more asymmetric centers. This disclosure includes the use of any of the optically pure stereoisomers as well as any combination of stereoisomers.

It is also intended that the Compounds of the Disclosure (PDE1 inhibitors) encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Disclosure may be replaced with deuterium (a stable isotope which is non-radioactive). Examples of known stable isotopes include, but not limited to, deuterium, 13 C, 15 N, 18 O. Alternatively, unstable isotopes, which are radioactive isotopes which contain additional neutrons compared to the abundant nuclides of the same species (i.e., element), e.g., 123I, 131I, 125I, 11C, 18F, may replace the corresponding abundant species of I, C and F. Another example of useful isotope of the compound of the disclosure is the 11C isotope. These radio isotopes are useful for radio-imaging and/or pharmacokinetic studies of the compounds of the disclosure.

The terms “treatment” and “treating” are to be understood accordingly as embracing treatment or amelioration of symptoms of disease as well as treatment of the cause of the disease.

For methods of treatment, the term “effective amount” is intended to encompass a therapeutically effective amount to treat breast cancer, e.g., inhibit the growth (volume or weight) of breast cancer when PDE-1 inhibitor and immune checkpoint inhibitor are administered in combination. The effective amount of PDE-1 inhibitor or immune checkpoint may be lower than if PDE-1 inhibitor or immune checkpoint is administered as a monotherapy.

The terms “patient” and “subject” include human or non-human (i.e., animal) patient, and are understood to be interchangeable within the context of this disclosure. In particular embodiment, the disclosure encompasses both human and nonhuman. In another embodiment, the disclosure encompasses nonhuman. In other embodiment, the term encompasses human.

The term “comprising” as used in this disclosure is intended to be open-ended and does not exclude additional, un-recited elements or method steps.

Dosages employed in practicing the present disclosure will of course vary depending, e.g., on the type of breast cancer to be treated, the particular PDE-1 inhibitors or immune checkpoint inhibitors, the mode of administration, and the therapy desired. PDE-1 inhibitors may be administered by any suitable route, including orally, parenterally (intravenously, intramuscular or subcutaneous), transdermally, or by inhalation, preferably administered orally. In certain embodiments, PDE-1 inhibitors, e.g., in depot formulation, are preferably administered parenterally, e.g., by injection. Immune checkpoint inhibitors may be administered by any suitable route, including orally, parenterally (intravenously, intramuscular or subcutaneous), transdermally, or by inhalation, preferably administered intravenously.

In general, satisfactory results, e.g., for the treatment of breast cancer are indicated to be obtained on oral administration of PDE-1 inhibitors at dosages of the order from about 0.01 to 2.0 mg/kg. In larger mammals, for example humans, an indicated daily dosage for oral administration of PDE-1 inhibitors will accordingly be in the range of from about 0.50 to 300 mg, conveniently administered once, or in divided doses 2 to 4 times, daily or in sustained release form. Unit dosage forms for oral administration thus for example may comprise from about 0.2 to 150 or 300 mg, e.g., from about 0.2 or 2.0 to 10, 25, 50, 75 100, 150, or 200 mg of PDE-1 inhibitors, together with a pharmaceutically acceptable diluent or carrier therefor.

PDE1 inhibitor and immune checkpoint inhibitor may be used in combination with one or more additional therapeutic agents, particularly at lower dosages than when the individual agents are used as a monotherapy so as to enhance the therapeutic activities of the combined agents without causing the undesirable side effects commonly occurring in conventional monotherapy. Therefore, PDE1 inhibitor and immune checkpoint inhibitor may be simultaneously, separately, sequentially, or contemporaneously administered with other agents useful in treating disease. In another example, side effects may be reduced or minimized by administering PDE1 inhibitor and immune checkpoint inhibitor in combination with one or more additional therapeutic agents in free or salt form, wherein the dosages of (i) the additional therapeutic agent(s) or (ii) PDE1 inhibitor and immune checkpoint inhibitor, are lower than if the agent/inhibitors are administered as a monotherapy.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

Pharmaceutical compositions may be prepared using conventional diluents or excipients and techniques known in the galenic art. Thus, oral dosage forms may include tablets, capsules, solutions, suspensions and the like.

### EXAMPLES—EFFECT OF PDE1 INHIBITOR TREATMENT ALONE AND IN COMBINATION WITH ANTI-PD-1 TREATMENT IN MOUSSE MODEL OF BREAST CANCER

### Example 1

### Materials and Methods

### In Vivo Tumor Implantation and Treatments

E0771 murine breast cancer cells are obtained from the American Type Culture Collection (ATCC) and maintained in DMEM medium containing 10% fetal bovine serum, 4 mM glutamine, 20 mM HEPES, and 1% penicillin/streptomycin. The cells are cultured in tissue culture flasks in a humidified incubator at 37° C., in an atmosphere of 5% CO₂ and 95% air. The E0771 tumor cells used for implantation are harvested and re-suspended at a concentration of 2.5×10⁶ cells/mL in cold PBS. Nine weeks old female C57Bl/6 mice (C57Bl/6J, Jackson Laboratory) are injected subcutaneously in the right flank with 0.5×10⁶ cells in 0.2 mL cold PBS. 10 days later the mice have a palpable size tumor, and specified size to start treatments (around 100 mm³). Mice are randomized in groups after the tumors are measured with a caliper in two dimensions to monitor size. Tumor size is calculated using the formula: Tumor Volume (mm³)=(w²×1)/2 where w=width and l=length, in mm. Tumors are measured twice per week for the duration of the study. Each mouse is euthanized as it reached a tumor volume endpoint of 1500 mm³ or if they show any type of discomfort, whichever comes first.

Mice are treated with Compound A alone, anti-PD-1 antibody alone, or the combination of Compound A and anti-PD-1 antibody. Compound A is synthesized at Intra-Cellular Therapies Inc. Compound A diets are used to treat mice. Compound A diet 900 ppm (diet supplements 900 mg of Compound A per kg to Picolab Rodent Diet 5053) is produced by Envigo (Madison WI). Mice are dosed 5 days a week with vehicle or Compound A.

The anti-PD-1 RMP1-14 (Lot No. 800121F12A) and isotype (Lot No. 749620N1) antibody are purchased from BioXCell. The stock solution is diluted in PBS to yield a dosing solution of 0.1 or 1 mg/mL, which delivered 1 or 10 mg/kg respectively in a dosing volume of 0.2 mL (10 mL/kg, for a 20 mg mouse). Mice are administered isotype or anti-PD-1 RMP1-14 intraperitoneally in 0.2 mL (10 mL/kg, for a 20 mg mouse) twice a week.

Animals are weighed twice per week until the completion of the study. Animals are observed frequently for signs of any toxicity and noteworthy clinical observations are recorded. Animal's body weight is monitored, and any animal with weight loss exceeding 15% for one measurement is euthanized.

### Flow Cytometry and Cell Sorting

Tumors are collected and minced in a digestion solution containing 2 mg/mL of collagenase D (Sigma-Aldrich) and 1 mg/mL DNase I (Sigma-Aldrich). The samples are incubated at 37° C. for 30-45 min and are passed through a 70-μm nylon cell strainer (Corning). The suspension is centrifuged at 1200 rpm for 3 min at 4° C. The pelleted cells are collected and resuspended in red blood cells lysis buffer (Sigma-Aldrich) and incubated at room temperature for 5 min, washed in PBS and centrifuged at 1200 rpm for 3 min at 4° C. Cells are resuspended in 2% Fc Block (BD Bioscience) and blocked for 30 min. Then fluorophore-conjugated primary antibodies are incubated in FACS buffer (1×HBSS (Thermo Fisher Scientific), 2% BSA and 0.5 mM EDTA) for 30 min at RT. The antibodies used for the macrophage panel are: PerCp-Cy5.5 anti-mouse CD45 (BD Bioscience), APC-Cy7 anti-mouse F4/80 (Invitrogen), PE anti-mouse CD11b (Biolegend), FITC anti-mouse INOS (BD Bioscience), BV510 anti-mouse CD80 (BD Bioscience), APC anti-mouse CD206 (Biolegend), and PE-Cy7 anti-mouse Arg1 (Invitrogen). The antibodies used for the T cells panel were: PerCp-Cy5.5 anti-mouse CD45 (BD Bioscience), APC-Cy7 anti-mouse CD3 (Biolegend), PE-Cy7 anti-mouse CD8 (BD Bioscience), BV610 anti-mouse CD4 (Biolegend), and BV786 anti-mouse NK1.1 (Biolegend). The antibodies used to sort the macrophages are: PerCp-Cy5.5 anti-mouse CD45 (BD Bioscience), APC anti-mouse F4/80 (Invitrogen), PE anti-mouse CD11b (Biolegend), and BV785 anti-mouse CD11c (Invitrogen). All antibodies are used at 1:200. After one wash in FACS buffer, cells are resuspended in FACS buffer and analyzed using the in house CytoFLEX (Beckman Coulter) or using a FACSAria II cell sorter (BD Biosciences) to sort and collect the macrophages (M1 and M2).

### RNAseg

Tumors are extracted with PureLINK RNA mini kit (Invitrogen), sorted macrophages are extracted with PicoPure RNA isolation kit (Thermo Fisher scientific), and quality and quantity are determined using an Agilent 2100 Bioanalyzer (Agilent Technologies). RNAseq libraries are prepared using the TrueSeq stranded mRNA kit for the tumors or the Clontech SMARTer Stranded Total RNA Seq Kit—Pico Input Mammalian for the sorted macrophages, respectively following the manufacturer's protocol. Libraries are purified using AMPure beads, pooled equimolarly, and run on a HiSeq 6000, paired end reads. FASTQ files are obtained. Reads are mapped to the mouse mm10 genome, and gene level and differential expression analysis is performed using the Rosalind tool. Genes with a p-value<0.05 and FC (fold-change)<−1.5 or >1.5 between conditions are determined to be differentially expressed. Heatmaps and pathways analysis are generated using Rosalind (https://www.rosalind.bio/).

### LC/MS/MS Quantification of Compound A

Tumor, liver, and feces samples are diluted in phosphate buffer and homogenized using ultrasonic processor. Plasma and homogenized tissue samples along with a set of standard and quality control samples are extracted by way of protein precipitation technique on Ostro plate (Waters) using 1% formic acid in acetonitrile spiked with internal standard. 2 μL of the filtered samples are injected using an autosampler and separated chromatographically using a Phenomenex, Synergi™, 2.5 m, 50×3 mm, Polar-RP column, isocratic mobile phase combination of 0.1% formic acid in water and 0.1% formic acid in acetonitrile (40/60) with flow rate of 0.8 mL/min. The Sciex Qtrap 6500 mass spectrometer is used as the detector with positive electrospray in MRM ionization mode and an ion source temperature of 650° C. Quantification of Compound A is carried out using a calibration curve established with standards for the corresponding analyte concentration, retention time and mass profile.

### Data Analysis.

Statistical analyses are performed using GraphPad Prism 9.1.0. Data are reported as mean±SD. In general, a two-tailed unpaired Student's t test is used when comparing two groups, with a p value<0.05 being considered significant and levels of significance denoted as *p<0.05. For in vivo experiments, n=number of animals. Samples are randomly allocated into experimental groups, and no data are excluded in the analysis.

### Results

The effect of PDE1 inhibitors alone or in combination with a sub-effective dose of programmed cell death-1 (PD-1) immune checkpoint inhibitors on tumor growth inhibition is investigated in a syngeneic mouse model of triple negative breast cancer. The effect of Compound A delivered in diet (900 ppm) is assessed on tumor growth as a monotherapy, or as a combination therapy in concert with anti-PD1 antibody (RMP1-14) at a 10 mg/kg dose.

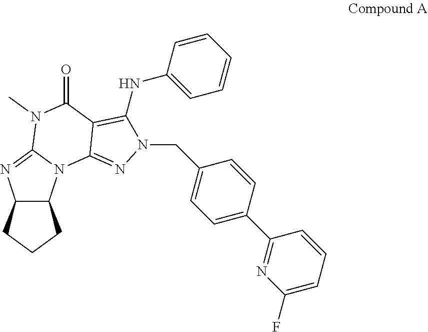 [SMILES: CN1C(=O)c2c(nn(Cc3ccc(-c4cccc(F)n4)cc3)c2Nc2ccccc2)N2C1=N[C@@H]1CCC[C@@H]12]

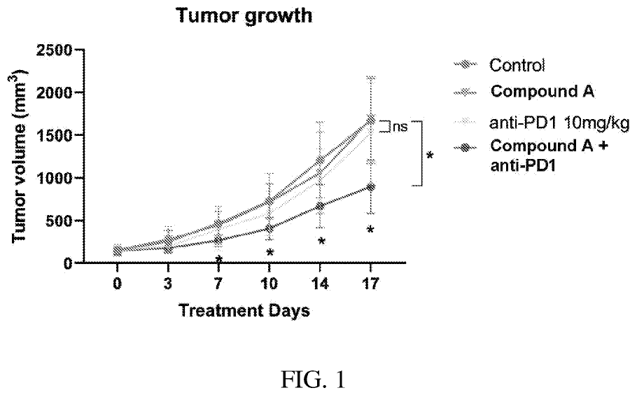

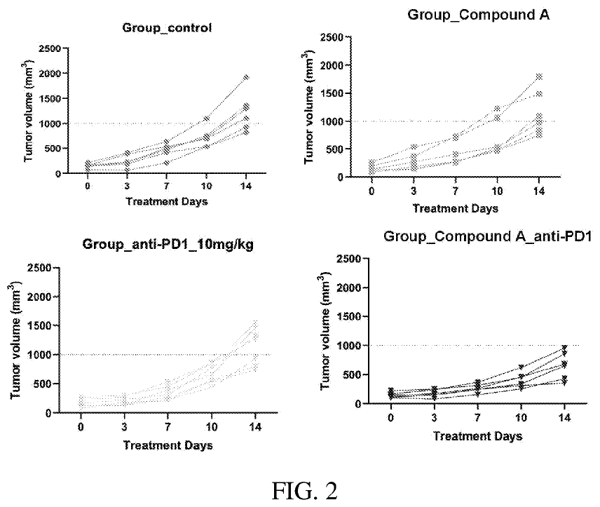

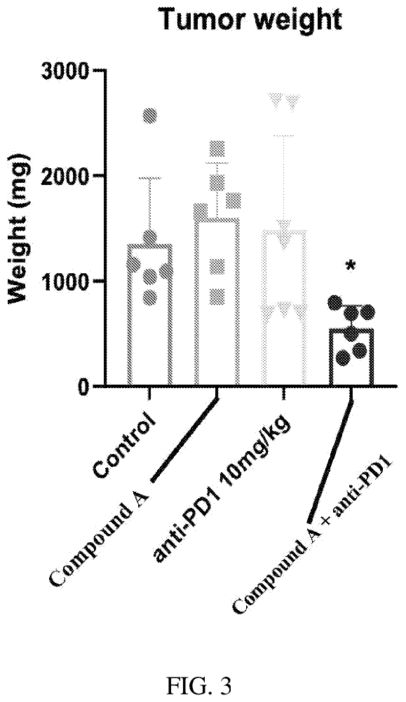

Neither Compound A (900 ppm) treatment alone nor anti-PD-1 antibody (10 mg/kg) treatment alone shows a significant effect on tumor volume at any given measurement time point (day 3, 7, 10, 14, and 17) (FIG. 1 and FIG. 2) or tumor weight at terminal sacrifice (treatment day 17) (FIG. 3). However, the combination of Compound A (900 ppm)+anti-PD1 antibody (10 mg/kg) treatments results in a significant reduction in tumor volume and tumor weight compared to isotype control (FIG. 1-3). No significant differences are observed in food intake or mouse weight between groups. Further, drug exposures for Compound A in plasma, tissues, and feces are also comparable for all groups receiving Compound A. These results demonstrate a synergistic ability of Compound A and anti-PD-1antibody to inhibit tumor growth in mouse model of breast cancer.

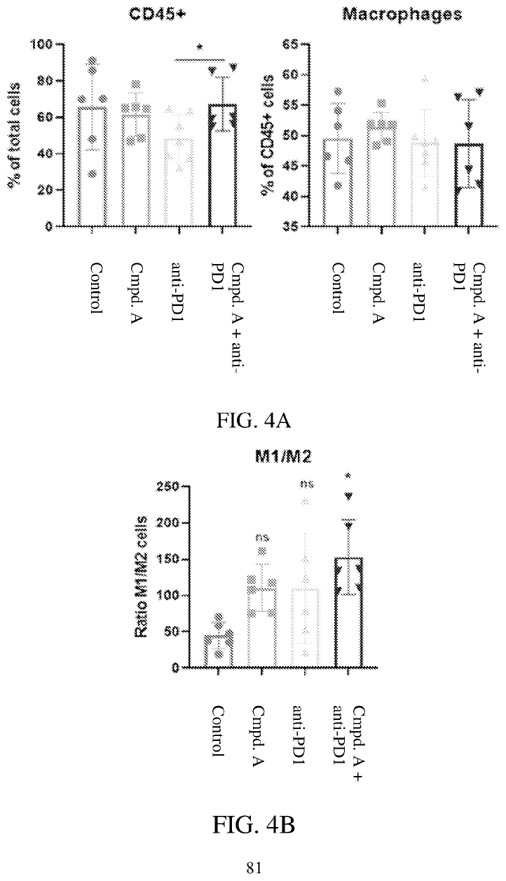

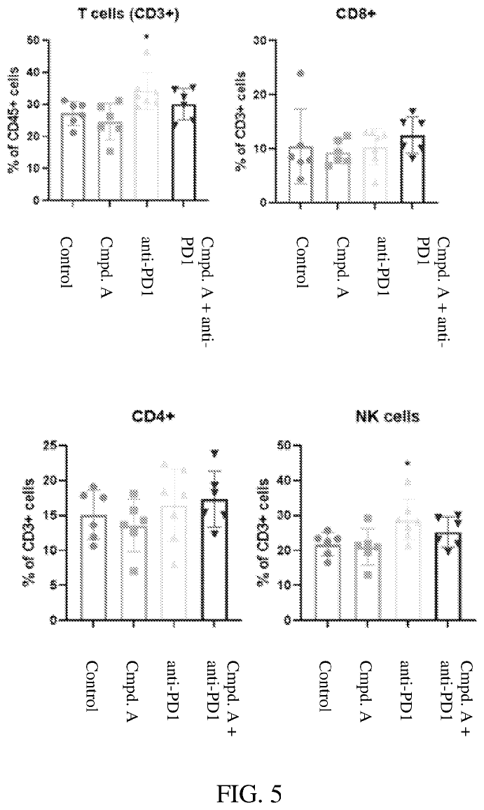

Next, tumors from isotype (control), Compound A (900 ppm) alone, anti-PD1 antibody (10 mg/kg) and Compound A (900 ppm)+anti-PD1 antibody (10 mg/kg) combination groups are analyzed by flow cytometry for drug-related changes of immune cells (macrophage, T-cell, and NK cells) in the tumor microenvironment (FIGS. 4 and 5). The results show that the Compound A+anti-PD1 antibody combination treatment does not alter the total number of macrophages in the tumor microenvironment (FIG. 4A), but the combination treatment has an effect on the ratio of M1/M2 (FIG. 4B). M1macrophages are pro-inflammatory, while M2 macrophages are anti-inflammatory. The Compound A+anti-PD1 antibody combination treatment significantly increases the ratio of M1/M2 macrophages compared to the isotype group (FIG. 4B). The combination treatment has no significant effect on T-cell populations (CD8+, CD4+) and NK cells, as measured by flow cytometry (FIG. 5).

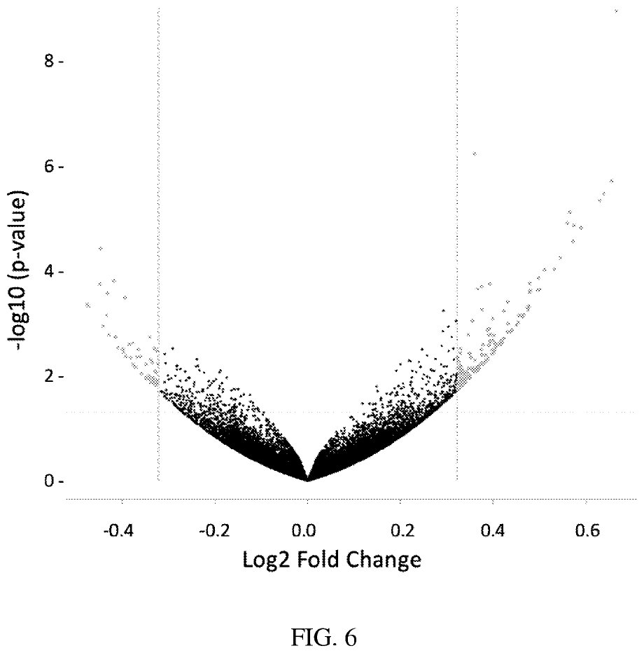

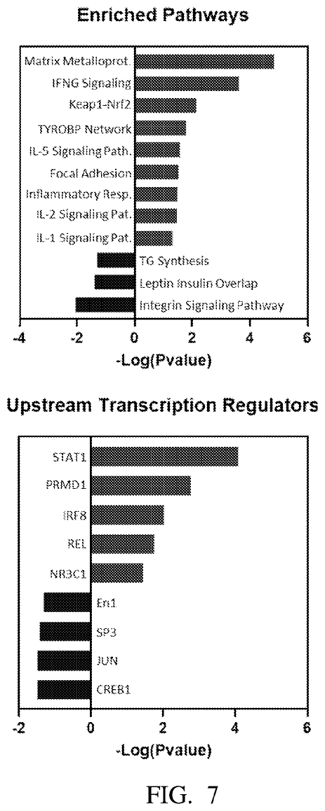

RNAseq analysis is performed to explore drug-related gene expression changes in Compound A+anti-PD1 (10 mg/kg) tumors compared with isotype (control) groups. Volcano plot of the comparison is shown in FIG. 6. The volcano plot shows that 48 genes are downregulated and 136 genes are upregulated in Compound A (900 ppm)+anti-PD1 (10 mg/kg) tumors (fold-change<−1.5 or >1.5; P<0.05). To characterize the role of these genes differentially expressed in Compound A (900 ppm)+anti-PD1 (10 mg/kg) tumors, pathway analysis is performed. Pathways enriched in the genes differentially expressed in Compound A (900 ppm)+anti-PD1 (10 mg/kg) tumors are shown in FIG. 7. Transcription regulators associated with upregulated or downregulated genes in Compound A+anti-PD1 (10 mg/kg) tumors are shown in FIG. 7. The RNAseq analysis shows that the combination therapy significantly downregulates genes involved in cell growth, survival, and migration pathways, while upregulating genes involved in inflammatory pathways. These results suggest that PDE-1 inhibitor in combination with anti-PD-1 antibody promotes antitumor immunity, leading to tumor growth inhibition.

### Example 2

### Materials and Methods

### In Vivo Tumor Implantation and Treatments

4T1 murine breast cancer cells are obtained from the American Type Culture Collection (ATCC) and maintained in RPMI medium containing 10% fetal bovine serum, 4 mM glutamine, and 1% penicillin/streptomycin. The cells were cultured in tissue culture flasks in a humidified incubator at 37° C., in an atmosphere of 5% CO2 and 95% air. The 4T1 tumor cells used for implantation are harvested and re-suspended at a concentration of 50×10³ cells/mL in cold PBS. Nine weeks old female Balb/c mice (Balb/cJ, Jackson Laboratory) are injected subcutaneously in the right flank with 10×10³ cells in 0.2 mL cold PBS. 10 days later the mice have a palpable size tumor, and specified size to start treatments (around 100 mm³). Mice are randomized in groups after the tumors are measured with a caliper in two dimensions to monitor size. Tumor size is calculated using the formula: Tumor Volume (mm³)=(w²×1)/2 where w=width and l=length, in mm. Tumors are measured twice per week for the duration of the study. Each mouse is euthanized as it reached a tumor volume endpoint of 1500 mm³ or if they show any type of discomfort, whichever comes first.

Mice are treated with Compound A alone, anti-PD-1 antibody alone, or the combination of Compound A and anti-PD-1 antibody. Compound A is synthesized at Intra-Cellular Therapies Inc. Compound A diets are used to treat mice. Compound A diet 300 ppm or 900 ppm (diet supplements 300 mg or 900 mg of Compound A per kg to Picolab Rodent Diet 5053) is produced by Envigo (Madison WI). Mice are dosed 5 days a week with vehicle or Compound A.

The anti-PD-1 RMP1-14 (Lot No. 800121F12A) and isotype (Lot No. 749620N1) antibody are purchased from BioXCell. The stock solution is diluted in PBS to yield a dosing solution of 0.1 or 1 mg/mL, which delivered 1 or 10 mg/kg respectively in a dosing volume of 0.2 mL (10 mL/kg, for a 20 mg mouse). Mice are administered isotype or anti-PD-1 RMP1-14 intraperitoneally in 0.2 mL (10 mL/kg, for a 20 mg mouse) twice a week.

Animals are weighed twice per week until the completion of the study. Animals are observed frequently for signs of any toxicity and noteworthy clinical observations are recorded. Animal's body weight is monitored, and any animal with weight loss exceeding 15% for one measurement is euthanized.

### LC/MS/MS Quantification of Compound A

Tumor, liver, and feces samples are diluted in phosphate buffer and homogenized using ultrasonic processor. Plasma and homogenized tissue samples along with a set of standard and quality control samples are extracted by way of protein precipitation technique on Ostro plate (Waters) using 1% formic acid in acetonitrile spiked with internal standard. 2 μL of the filtered samples are injected using an autosampler and separated chromatographically using a Phenomenex, Synergi™, 2.5 m, 50×3 mm, Polar-RP column, isocratic mobile phase combination of 0.1% formic acid in water and 0.1% formic acid in acetonitrile (40/60) with flow rate of 0.8 mL/min. The Sciex Qtrap 6500 mass spectrometer is used as the detector with positive electrospray in MRM ionization mode and an ion source temperature of 650° C. Quantification of Compound A is carried out using a calibration curve established with standards for the corresponding analyte concentration, retention time and mass profile.

### Data Analysis.

Statistical analyses are performed using GraphPad Prism 9.1.0. Data are reported as mean±SD. In general, a two-tailed unpaired Student's t test is used when comparing two groups, with a p value<0.05 being considered significant and levels of significance denoted as *p<0.05; **p<0.01; and ***p<0.001. For in vivo experiments, n=number of animals. Samples are randomly allocated into experimental groups, and no data are excluded in the analysis.

### Results

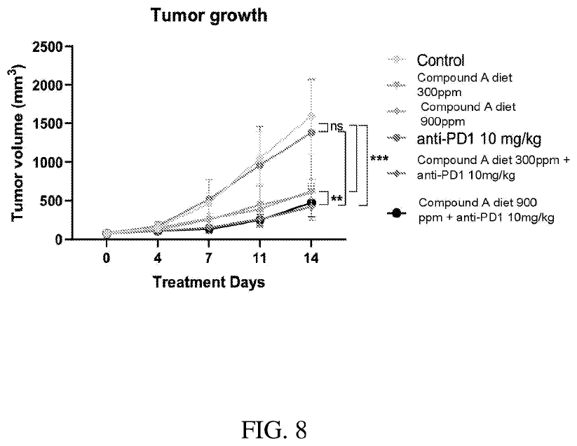

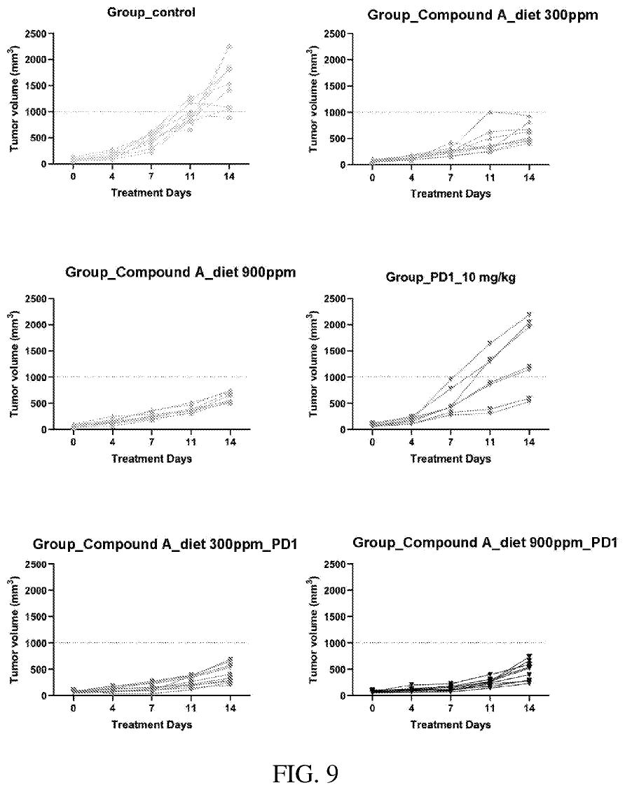

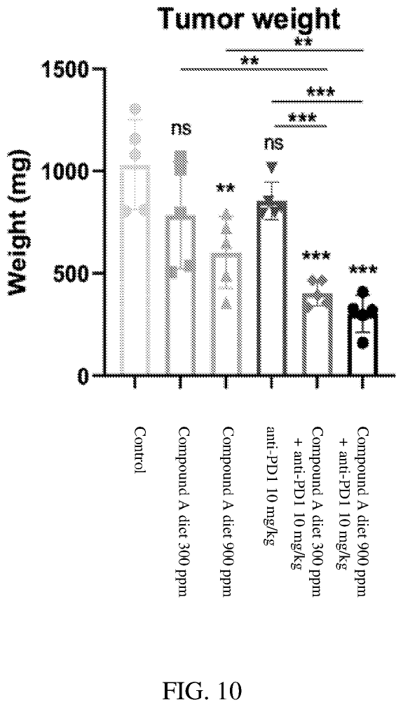

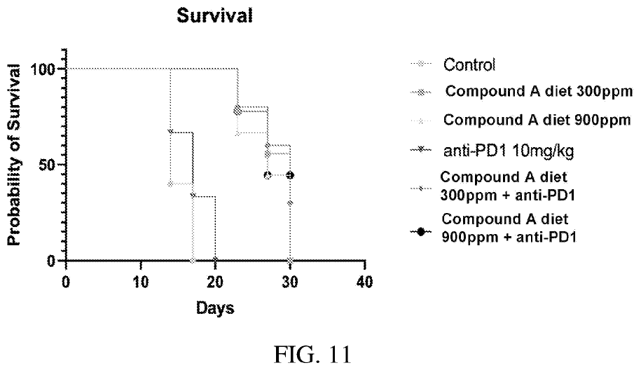

The effect of Compound A delivered in diet (300 ppm or 900 ppm) is assessed on growth of 4T1 tumors as a monotherapy, or as a combination therapy in concert with anti-PD1 antibody (RMP1-14) at a 10 mg/kg dose. Tumor volumes (FIG. 8 and FIG. 9) and tumor weights (FIG. 10) for the anti-PD1 (10 mg/kg) group are not different from isotype (control) at any given measurement time point or at terminal sacrifice (treatment day 14). Treatment with different Compound A monotherapy doses or in different combinations with anti-PD1, however, significantly reduce tumor volume or weight relative to isotype (control), as shown in FIG. 8-10. Survival of animals are also improved in the monotherapy and combination group (FIG. 11). Compound A exposure in plasma and tissues is comparable in groups receiving drug in the diet in absence or presence of anti-PD1.

### Example 3

### Materials and Methods

### In Vivo Tumor Implantation and Treatments

E0771 murine breast cancer cells are obtained from the American Type Culture Collection (ATCC) and maintained in DMEM medium containing 10% fetal bovine serum, 4 mM glutamine, 20 mM HEPES, and 1% penicillin/streptomycin. The cells are cultured in tissue culture flasks in a humidified incubator at 37° C., in an atmosphere of 5% CO₂ and 95% air. The E0771 tumor cells used for implantation are harvested and re-suspended at a concentration of 2.5×10⁶ cells/mL in cold PBS. Nine weeks old female C₅₇Bl/6 mice (C₅₇Bl/6J, Jackson Laboratory) are injected subcutaneously in the right flank with 0.5×10⁶ cells in 0.2 mL cold PBS. 10 days later the mice have a palpable size tumor, and specified size to start treatments (around 100 mm³). Mice are randomized in groups after the tumors are measured with a caliper in two dimensions to monitor size. Tumor size is calculated using the formula: Tumor Volume (mm³)=(w²×1)/2 where w=width and l=length, in mm. Tumors are measured twice per week for the duration of the study. Each mouse is euthanized as it reached a tumor volume endpoint of 1500 mm³ or if they show any type of discomfort, whichever comes first.

Mice are treated with Compound B alone, anti-PD-1 antibody alone, or the combination of Compound B and anti-PD-1 antibody. Compound B is synthesized at Intra-Cellular Therapies Inc. Compound B diets are used to treat mice. Compound B diet 100 ppm, 300 ppm, or 900 ppm (diet supplements 100 mg, 300 mg, or 900 mg of Compound B per kg to Picolab Rodent Diet 5053) is produced by Envigo (Madison WI). Mice are dosed 5 days a week with vehicle or Compound B.

The anti-PD-1 RMP1-14 (Lot No. 800121F12A) and isotype (Lot No. 749620N1) antibody are purchased from BioXCell. The stock solution is diluted in PBS to yield a dosing solution of 0.1 or 1 mg/mL, which delivered 1 or 10 mg/kg respectively in a dosing volume of 0.2 mL (10 mL/kg, for a 20 mg mouse). Mice are administered isotype or anti-PD-1 RMP1-14 intraperitoneally in 0.2 mL (10 mL/kg, for a 20 mg mouse) twice a week.

Animals are weighed twice per week until the completion of the study. Animals are observed frequently for signs of any toxicity and noteworthy clinical observations are recorded. Animal's body weight is monitored, and any animal with weight loss exceeding 15% for one measurement is euthanized.

### LC/MS/MS Quantification of Compound B

Tumor, liver, and feces samples are diluted in phosphate buffer and homogenized using ultrasonic processor. Plasma and homogenized tissue samples along with a set of standard and quality control samples are extracted by way of protein precipitation technique on Ostro plate (Waters) using 1% formic acid in acetonitrile spiked with internal standard. 2 μL of the filtered samples are injected using an autosampler and separated chromatographically using a Phenomenex, Synergi™, 2.5 m, 50×3 mm, Polar-RP column, isocratic mobile phase combination of 0.1% formic acid in water and 0.1% formic acid in acetonitrile (40/60) with flow rate of 0.8 mL/min. The Sciex Qtrap 6500 mass spectrometer is used as the detector with positive electrospray in MRM ionization mode and an ion source temperature of 650° C. Quantification of Compound B is carried out using a calibration curve established with standards for the corresponding analyte concentration, retention time and mass profile.

### Data Analysis.

Statistical analyses were performed using GraphPad Prism 9.1.0 for PC. Data are reported as mean±SD. In general, a two-tailed unpaired Student's t test was used when comparing two groups, with a p value<0.05 being considered significant and levels of significance denoted as *p<0.05; **p<0.01; and ***p<0.001. For in vivo experiments, n=number of animals. Samples were randomly allocated into experimental groups, and no data were excluded in the analysis.

### Results

The effect of Compound B delivered in diet (100 ppm, 300 ppm, or 900 ppm) is assessed on growth of E0771 tumors as a monotherapy, or as a combination therapy in concert with anti-PD1 antibody (RMP1-14) at a 1 mg/kg dose.

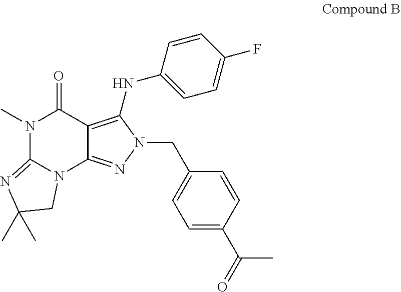 [SMILES: CC(=O)c1ccc(Cn2nc3c(c2Nc2ccc(F)cc2)C(=O)N(C)C2=NC(C)(C)CN23)cc1]

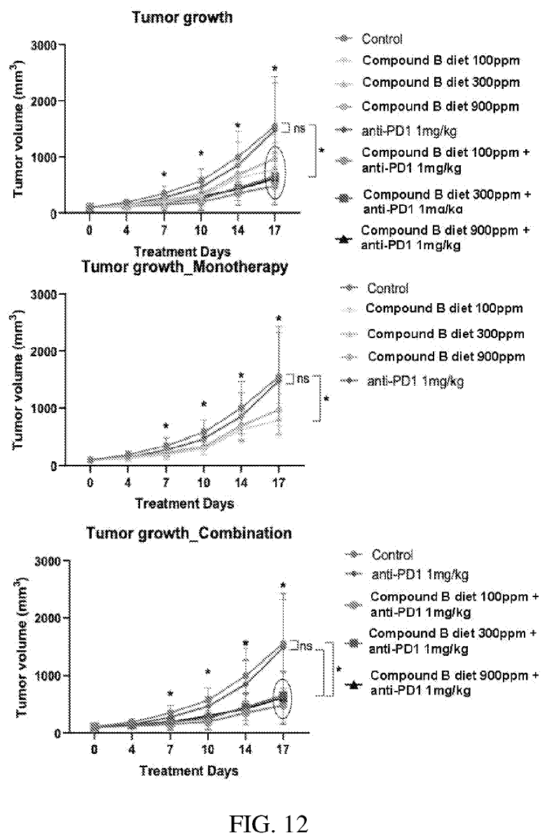

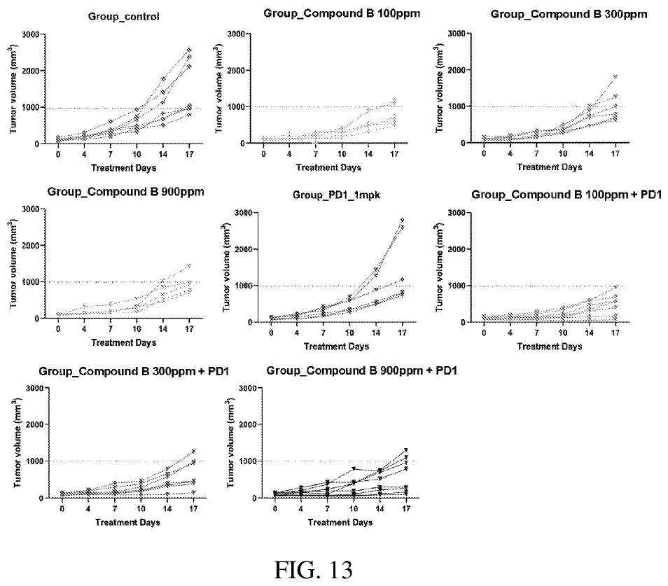

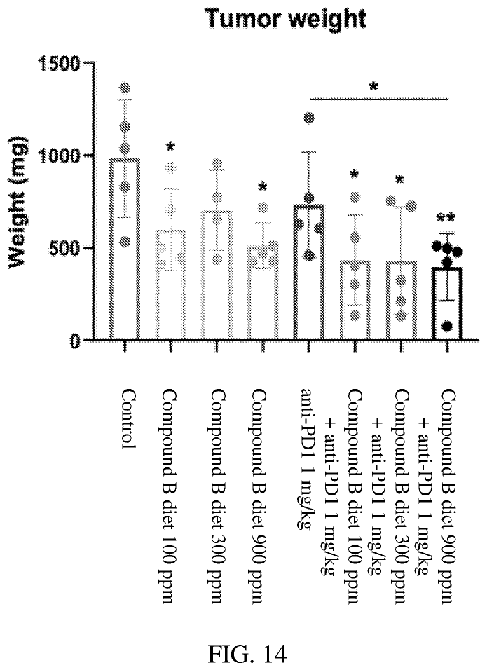

Tumor volumes (FIG. 12 and FIG. 13) and tumor weights (FIG. 14) in mice treated with anti-PD1 (1 mg/kg) alone are not different from isotype (control) at any given measurement time point or at terminal sacrifice (treatment day 17). Treatment with different dose levels of Compound B as monotherapy or in combination with anti-PD1, however, significantly reduces tumor volume or weight relative to isotype (control) and/or anti-PD1 alone, as shown in FIG. 12-14. Compound B exposure in plasma and tissues is comparable in groups receiving drug in the diet in absence or presence of anti-PD1.

### Example 4

### Materials and Methods

### In Vivo Tumor Implantation and Treatments

4T1 murine breast cancer cells are obtained from the American Type Culture Collection (ATCC) and maintained in RPMI medium containing 10% fetal bovine serum, 4 mM glutamine, and 1% penicillin/streptomycin. The cells were cultured in tissue culture flasks in a humidified incubator at 37° C., in an atmosphere of 5% CO2 and 95% air. The 4T1 tumor cells used for implantation are harvested and re-suspended at a concentration of 50×10³ cells/mL in cold PBS. Nine weeks old female Balb/c mice (Balb/cJ, Jackson Laboratory) are injected subcutaneously in the right flank with 10×10³ cells in 0.2 mL cold PBS. 10 days later the mice have a palpable size tumor, and specified size to start treatments (around 100 mm³). Mice are randomized in groups after the tumors are measured with a caliper in two dimensions to monitor size. Tumor size is calculated using the formula: Tumor Volume (mm³) (w²×1)/2 where w=width and l=length, in mm. Tumors are measured twice per week for the duration of the study. Each mouse is euthanized as it reached a tumor volume endpoint of 1500 mm³ or if they show any type of discomfort, whichever comes first.

Mice are treated with Compound B alone, anti-PD-1 antibody alone, or the combination of Compound B and anti-PD-1 antibody. Compound B is synthesized at Intra-Cellular Therapies Inc. Compound B diets are used to treat mice. Compound B diet 100 ppm, 300 ppm, or 900 ppm (diet supplements 100 mg, 300 mg, or 900 mg of Compound B per kg to Picolab Rodent Diet 5053) is produced by Envigo (Madison WI). Mice are dosed 5 days a week with vehicle or Compound B.

The anti-PD-1 RMP1-14 (Lot No. 800121F12A) and isotype (Lot No. 749620N1) antibody are purchased from BioXCell. The stock solution is diluted in PBS to yield a dosing solution of 0.1 or 1 mg/mL, which delivered 1 or 10 mg/kg respectively in a dosing volume of 0.2 mL (10 mL/kg, for a 20 mg mouse). Mice are administered isotype or anti-PD-1 RMP1-14 intraperitoneally in 0.2 mL (10 mL/kg, for a 20 mg mouse) twice a week.

Animals are weighed twice per week until the completion of the study. Animals are observed frequently for signs of any toxicity and noteworthy clinical observations are recorded. Animal's body weight is monitored, and any animal with weight loss exceeding 15% for one measurement is euthanized.

### Flow Cytometry and Cell Sorting

Flow cytometry and cell sorting are performed substantially in accordance with Example 1.

### RNAseq

RNAseq is performed substantially in accordance with Example 1.

### LC/MS/MS Quantification of Compound B

Tumor, liver, and feces samples are diluted in phosphate buffer and homogenized using ultrasonic processor. Plasma and homogenized tissue samples along with a set of standard and quality control samples are extracted by way of protein precipitation technique on Ostro plate (Waters) using 1% formic acid in acetonitrile spiked with internal standard. 2 μL of the filtered samples are injected using an autosampler and separated chromatographically using a Phenomenex, Synergi™, 2.5 m, 50×3 mm, Polar-RP column, isocratic mobile phase combination of 0.1% formic acid in water and 0.1% formic acid in acetonitrile (40/60) with flow rate of 0.8 mL/min. The Sciex Qtrap 6500 mass spectrometer is used as the detector with positive electrospray in MRM ionization mode and an ion source temperature of 650° C. Quantification of Compound A is carried out using a calibration curve established with standards for the corresponding analyte concentration, retention time and mass profile.

Data Analysis.

Statistical analyses are performed using GraphPad Prism 9.1.0. Data are reported as mean±SD. In general, a two-tailed unpaired Student's t test is used when comparing two groups, with a p value<0.05 being considered significant and levels of significance denoted as *p<0.05; **p<0.01; and ***p<0.001. For in vivo experiments, n=number of animals. Samples are randomly allocated into experimental groups, and no data are excluded in the analysis.

### Results

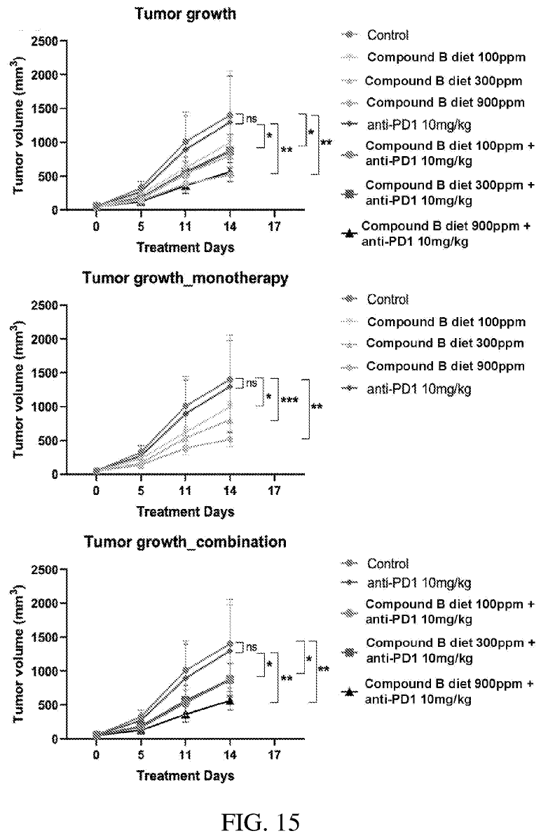

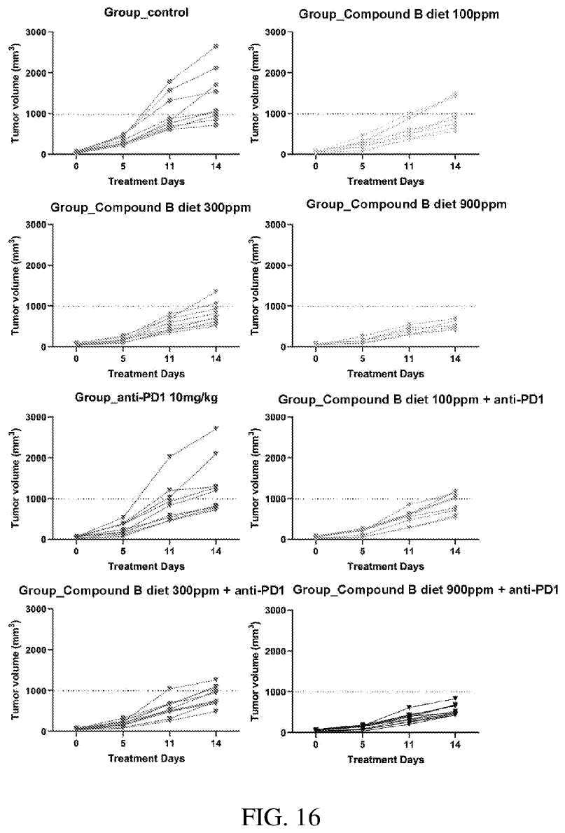

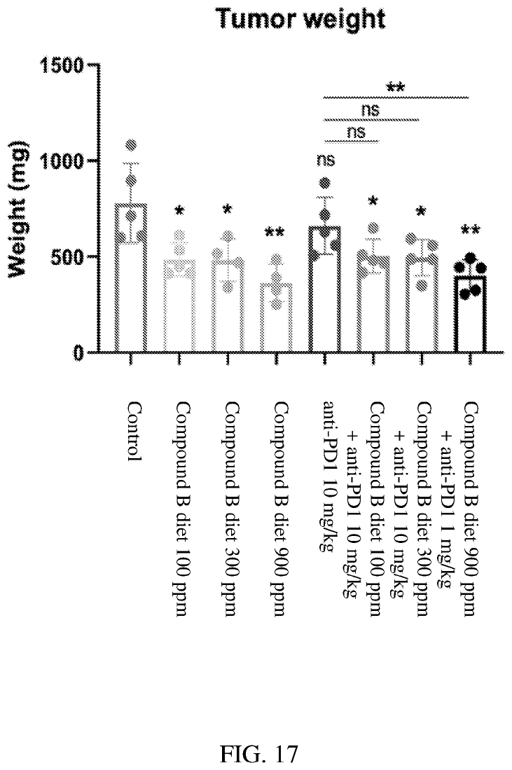

The effect of Compound B delivered in diet (100 ppm, 300 ppm or 900 ppm) is assessed on growth of 4T1 tumors as a monotherapy, or as a combination therapy in concert with anti-PD1 antibody (RMP1-14) at a 10 mg/kg dose. Tumor volumes (FIG. 15 and FIG. 16) and tumor weights (FIG. 17) in mice treated with anti-PD1 (10 mg/kg) alone are not different from isotype (control) during the experiment after day 5 or at terminal sacrifice (treatment day 14). Treatment with different doses of Compound B as monotherapy or in combination with anti-PD1, however, significantly reduces tumor volume and weight relative to isotype (control) and/or anti-PD1 alone, as shown in FIG. 15-17. Compound B exposure in plasma and tissues is comparable in groups receiving drug in the diet in absence or presence of anti-PD1.

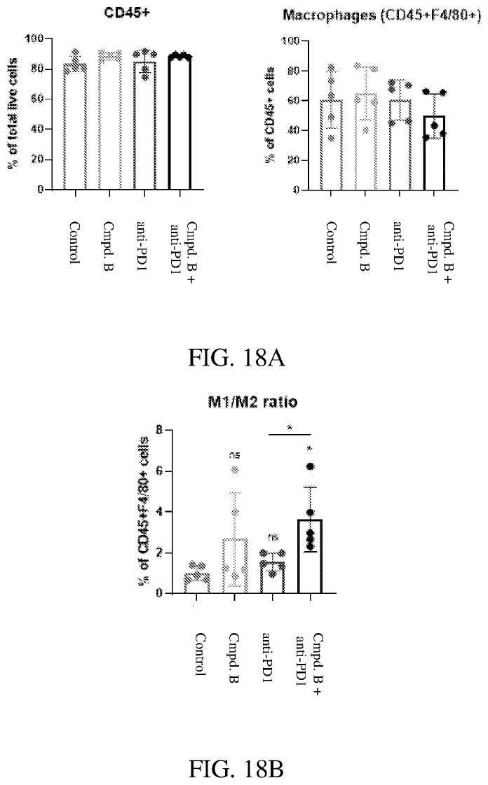

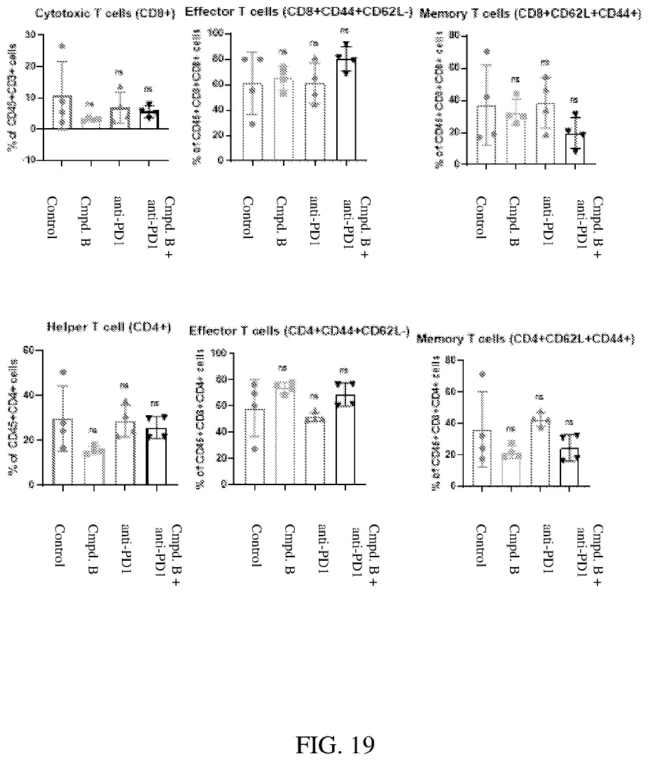

Next, tumors from isotype (control), Compound B (900 ppm) alone, anti-PD1 antibody (10 mg/kg) alone, and Compound B (900 ppm)+anti-PD1 antibody (10 mg/kg) combination groups are analyzed by flow cytometry for drug-related changes of immune cells (macrophage, T-cell, and NK cells (in the tumor microenvironment (FIGS. 18 and 19). The results show that the Compound B+anti-PD1 antibody combination treatment does not alter the total number of macrophages in the tumor microenvironment (FIG. 18A), but the combination treatment has an effect on the ratio of M1/M2 (FIG. 18B). M1 macrophages are pro-inflammatory, while M2 macrophages are anti-inflammatory. The Compound B+anti-PD1 antibody combination treatment significantly increases the ratio of M1/M2 macrophages compared to the isotype group (FIG. 18B). The combination treatment has no significant effect on T-cell populations, as measured by flow cytometry (FIG. 19).

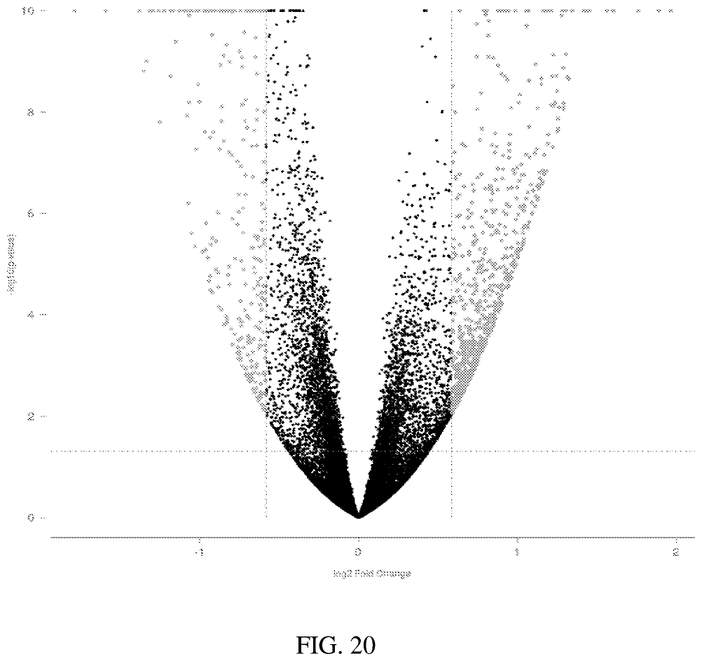

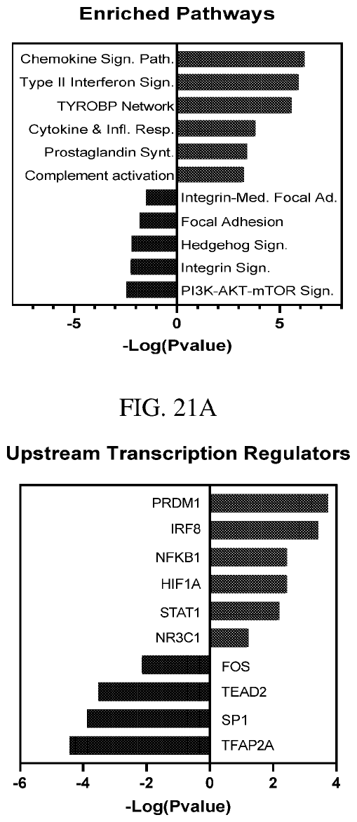

RNAseq analysis is performed to explore drug-related gene expression changes in Compound B (900 ppm)+anti-PD1 (10 mg/kg) tumors compared with isotype (control) groups. Volcano plot of the comparison is shown in FIG. 20. The volcano plot shows that 708 genes are upregulated and 281 genes are downregulated in Compound B (900 ppm)+anti-PD1 (10 mg/kg) tumors (fold-change<−1.5 or >1.5; P<0.05). To characterize the role of these genes differentially expressed in Compound B (900 ppm)+anti-PD1 (10 mg/kg) tumors, pathway analysis is performed. Pathways enriched (including genes regulating inflammatory processes, such as chemokine signaling pathways, including Type II interferon signaling or cytokine and inflammatory response) and pathways downregulated (including those involved in cellular proliferation, survival, and migration pathways) in the genes differentially expressed in Compound B+anti-PD1 (10 mg/kg) tumors are shown in FIG. 21A. Transcription regulators associated with upregulated or downregulated genes in Compound B (900 ppm)+anti-PD1 (10 mg/kg) tumors are shown in FIG. 21B. The RNAseq analysis shows that the combination therapy significantly downregulates genes involved in cell growth, survival, and migration pathways (TFAP2A, SP1, TEAD2, and FOS), while upregulating genes involved in inflammatory pathways (PRDM1, IRF8, NFKβ1, HIF1A, STAT1, and NR3C1). These results suggest that Compound B in combination with anti-PD1 antibody promotes antitumor immunity, leading to tumor growth inhibition.

1. A method of treating breast cancer comprising administering a pharmaceutically acceptable amount of a PDE1 inhibitor alone or in combination with a pharmaceutically acceptable amount of an immune checkpoint inhibitor to a subject in need thereof.

2. The method according to claim 1, wherein the breast cancer is triple-negative breast cancer (TNBC).

3. The method according to claim 2, wherein the TNBC is a high-risk early stage TNBC.

4. The method according to claim 2, wherein the treatment is an adjuvant treatment after the TNBC is removed by surgery.

5. The method according to claim 2, wherein the subject has locally recurrent unresectable or metastatic TNBC whose tumors express PD-L1 (Combined Positive Score (CPS)≥1).

6. The method according to claim 1, wherein the immune checkpoint inhibitor is selected from one or more of inhibitors of CTLA-4, PD-1 and/or PD-L1.

7. The method according to claim 1, wherein the immune checkpoint inhibitor is an inhibitor of PD-1.

8. The method according to claim 1, wherein the immune checkpoint inhibitor is an anti-PD-1 antibody.

9. The method according to claim 1, wherein the immune checkpoint inhibitor comprises one or more members selected from nivolumab, pembrolizumab, cemiplimab, ipilimumab, avelumab, durvalumab, atezolizumab, and spartalizumab.

10. The method according to claim 1, wherein the subject is suffering from a systemic inflammatory response, a gastrointestinal inflammation-related disorder, an endocrine inflammation-related disorder, a dermatologic inflammation-related disorder, an ophthalmologic inflammation-related disorder, a neurologic inflammation-related disorder, a hematologic inflammation-related disorder, a genitourinary inflammation-related disorder, a respiratory inflammation-related disorder, a musculoskeletal inflammation-related disorder, a cardiac inflammation-related disorder, or a defined systemic inflammation-related disorder.

11. The method according to claim 1, wherein the administration of the pharmaceutically acceptable amount of the PDE1 inhibitor alone or in combination with the pharmaceutically acceptable amount of the immune checkpoint inhibitor to the subject increases the M1/M2 ratio of macrophages in the tumor microenvironment.

12. A method of prophylaxis or mitigation of a disease, disorder or adverse effect consequent to administration of an immune checkpoint inhibitor therapy to a subject suffering from breast cancer, the method comprising reducing the amount of checkpoint inhibitor administered to the subject and administering a pharmaceutically acceptable amount of a PDE1 inhibitor in combination with the immune checkpoint inhibitor therapy to the subject.

13. The method according to claim 12, wherein the breast cancer is triple-negative breast cancer (TNBC).

14. The method according to claim 12, wherein the checkpoint inhibitor is selected from one or more of inhibitors of CTLA-4, PD-1 and/or PD-L1.

15. The method according to claim 12, wherein the immune checkpoint inhibitor is an anti-PD-1 antibody.

16. The method according to claim 1, wherein the PDE1 inhibitor is a compound selected from
(A) Formula I: 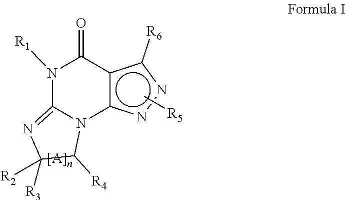
wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl;
or R₅ is attached to one of the nitrogens on the pyrazolo portion of Formula I and is a moiety of Formula A 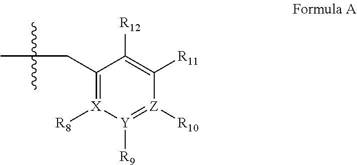
wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present;
(iv) R₆ is H, alkyl, aryl, heteroaryl, arylalkyl (e.g., benzyl), arylamino (e.g., phenylamino), heteroarylamino, N,N-dialkylamino, N,N-diarylamino, or N-aryl-N-(arylalkyl)amino (e.g., N-phenyl-N-(1,1′-biphen-4-ylmethyl)amino);
(v) n=0 or 1; and
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates;
(B) Formula II: 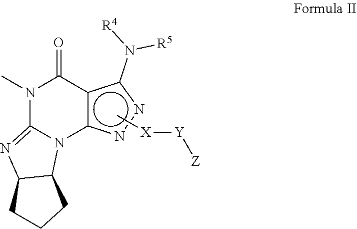
(i) X is C₁₋₆alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), C(O) R¹, N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹ is C₁₋₆alkyl, haloC₁₋₆alkyl, OH or OC₁₋₆alkyl (e.g., OCH₃);
(v) R² and R³ are independently H or C₁₋₆alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy; and
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl);
in free, salt or prodrug form, including its enantiomers, diastereoisomers and racemates;
(C) Formula III: 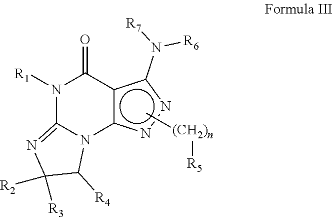
wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4;
in free, salt or prodrug form, including its enantiomers, diastereoisomers and racemates;
(D) Formula IV 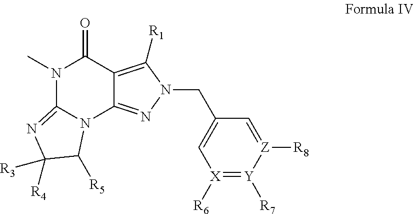
wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively); and
(iv) R₆, R₇ and R₈ are independently:
H,
C₁₋₄alkyl (e.g., methyl),
pyrid-2-yl substituted with hydroxy, or
—S(O)₂—NH₂;
provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy;
in free, salt or prodrug form, including its enantiomers, diastereoisomers and racemates;
(E) Formula 1a: 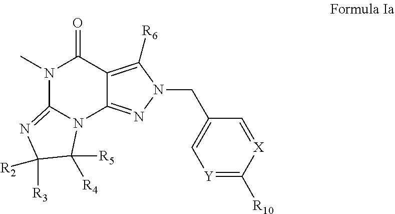
wherein
(i) R₂ and R₅ are independently H or hydroxy and R₃ and R₄ together form a tri- or tetramethylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino; and
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
X and Y are independently C or N;
in free, pharmaceutically acceptable salt or prodrug form, including its enantiomers, diastereoisomers and racemates;
(F) Formula V 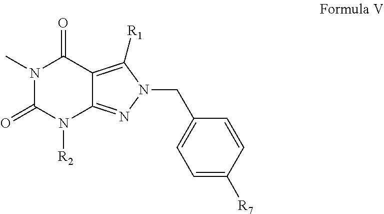
wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl, isobutyl or neopentyl); and
(iii) R₃ is —SO₂NH₂ or —COOH;
in free, salt or prodrug form, including its enantiomers, diastereoisomers and racemates; and/or
(G) Formula V 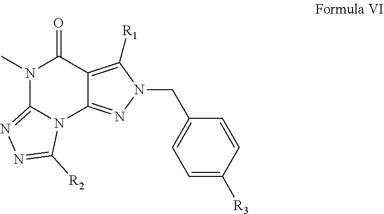
wherein
(i) R₁ is —NH(R₄), wherein R₄ is phenyl optionally substituted with halo (e.g., fluoro), for example, 4-fluorophenyl;
(ii) R₂ is H or C₁₋₆alkyl (e.g., methyl or ethyl); and
(iii) R₃ is H, halogen (e.g., bromo), C₁₋₆alkyl (e.g., methyl), aryl optionally substituted with halogen (e.g., 4-fluorophenyl), heteroaryl optionally substituted with halogen (e.g., 6-fluoropyrid-2-yl or pyrid-2-yl), or acyl (e.g., acetyl);
in free, salt or prodrug form, including its enantiomers, diastereoisomers and racemates.

17. The method according to any claim 16, wherein the PDE1 inhibitor is selected from any of the following 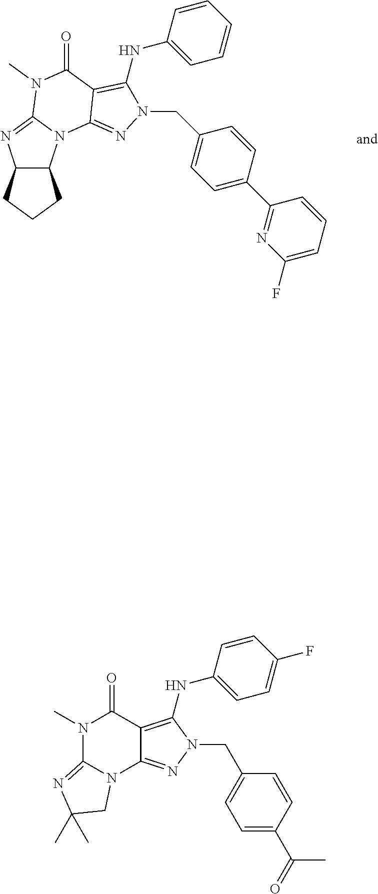 [SMILES: CN1C(=O)c2c(nn(Cc3ccc(-c4cccc(F)n4)cc3)c2Nc2ccccc2)N2C1=N[C@@H]1CCC[C@@H]12 ; CC(=O)c1ccc(Cn2nc3c(c2Nc2ccc(F)cc2)C(=O)N(C)C2=NC(C)(C)CN23)cc1]
in free or pharmaceutically acceptable salt form.

18. The method according to claim 16, wherein the PDE1 inhibitor is 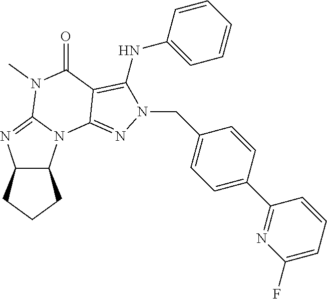 [SMILES: CN1C(=O)c2c(nn(Cc3ccc(-c4cccc(F)n4)cc3)c2Nc2ccccc2)N2C1=N[C@@H]1CCC[C@@H]12]
in free or pharmaceutically acceptable salt form, e.g., in monophosphate salt form.

19. The method according to claim 16, wherein the PDE1 inhibitor is 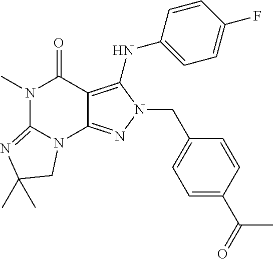 [SMILES: CC(=O)c1ccc(Cn2nc3c(c2Nc2ccc(F)cc2)C(=O)N(C)C2=NC(C)(C)CN23)cc1]
in free or pharmaceutically acceptable salt form.

20. A pharmaceutical combination therapy comprising a pharmaceutically acceptable amount of a PDE1 inhibitor and a pharmaceutically acceptable amount of an immune checkpoint inhibitor for use in the method of claim 1.
